(19) United States
(12) Patent Application Publication
(10) Pub. No.: US 2010/0158837 A1
(43) Pub. Date: Jun. 24, 2010

(54) IRON OXIDE-BINDING PEPTIDES

(75) Inventors: STEPHEN R. FAHNESTOCK, Wilmington, DE (US); Kristy N. Kostichka, Wilmington, DE (US); Anju Parthasarathy, Glenmoore, PA (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/632,827

(22) Filed: Dec. 8, 2009

Related U.S. Application Data
(60) Provisional application No. 61/138,623, filed on Dec. 18, 2008.

(51) Int. Cl. A61K 8/64; A61Q 17/04; C07K 14/00; C07K 7/08; A61Q 1/02; A61Q 3/02; A61Q 5/06

(52) U.S. Cl. 424/61; 424/63; 514/6; 514/14; 514/13; 530/326; 530/327; 530/350; 424/70.6

(57) ABSTRACT

Peptides having strong affinity for iron oxide pigment particles have been identified. Peptide-based reagents comprising at least one of the present iron oxide-based pigment-binding peptides and at least one body surface-binding peptide are described. The peptide-based reagents may be used in conjunction with at least one iron oxide-based pigment to color body surfaces.

### CROSS-REFERENCE TO A RELATED APPLICATION

This application claims the benefit of U.S. Provisional Patent Application No. 61/138,623 filed Dec. 18, 2008, incorporated herein by reference.

### FIELD OF THE INVENTION

The invention relates to the field of personal care products. More specifically, the invention relates to peptide-based reagents comprising at least one body surface-binding peptide and at least one of the present iron oxide-based pigment-binding peptides as well personal care compositions comprising such materials. A method of coloring a body surface using one of the present peptide-based reagents in combination with an iron oxide-based pigment is also provided.

### BACKGROUND OF THE INVENTION

Iron oxides are used as pigments in a variety of personal care product coloring applications due to their wide range of colors (such as reds, yellows, browns, and blacks), stability to degradation, and their non-toxic nature. Coloring body surfaces using iron oxide-based pigments is a less-toxic alternative to colorants such as oxidative hair dyes and/or colorants requiring covalent attachment to the body surface. However, coloring body surfaces non-covalently with iron oxide-based pigments suffers in a lack of color durability.

There have been numerous attempts to enhance the binding of cosmetic agents, including coloring agents, to body surfaces such as hair, skin, and nails using. For example, Richardson et al. in U.S. Pat. No. 5,490,980 and Green et al. in U.S. Pat. No. 6,267,957 describe the covalent attachment of cosmetic agents, such as skin conditioners, hair conditioners, coloring agents, sunscreens, and perfumes, to hair, skin, and nails using the enzyme transglutaminase. This enzyme crosslinks an amine moiety on the cosmetic agent to the glutamine residues in skin, hair, and nails. Similarly, WO 01/07009 to Green et al. describes the use of the enzyme lysine oxidase to covalently attach cosmetic agents to hair, skin, and nails.

In another approach, cosmetic agents have been covalently attached to proteins or protein hydrolysates. For example, U.S. Pat. No. 5,192,332 to Lang et al. describes temporary coloring compositions that contain an animal or vegetable protein, or hydrolysate thereof, which contain residues of dye molecules grafted onto the protein chain. In those compositions, the protein serves as a conditioning agent and does not enhance the binding of the cosmetic agent to hair, skin, or nails. Horikoshi et al. in JP 08104614 and Igarashi et al. in U.S. Pat. No. 5,597,386 describe hair coloring agents that consist of an anti-keratin antibody covalently attached to a dye or pigment. The antibody binds to the hair, thereby enhancing the binding of the hair coloring agent to the hair. Similarly, JP 09003100 to Kizawa et al. describes an antibody that recognizes the surface layer of hair and its use to treat hair. A hair coloring agent consisting of that anti-hair antibody coupled to colored latex particles is also described. The use of antibodies to enhance the binding of dyes to the hair is effective in increasing the durability of the hair coloring, but these antibodies are difficult and expensive to produce.

Terada et al. in JP 2002363026 describe the use of conjugates consisting of single-chain antibodies, preferably anti-keratin antibodies, coupled to dyes, ligands, and cosmetic agents for skin and hair care compositions. The single-chain antibodies may be prepared using genetic engineering techniques, but are still difficult and expensive to prepare because of their large size. WO 00/048558 to Findlay describes the use of calycin proteins, such as β-lactoglobulin, which contain a binding domain for a cosmetic agent and another binding domain that binds to at least a part of the surface of a hair fiber or skin surface, for conditioners, dyes, and perfumes. Again these proteins are large and difficult and expensive to produce.

Peptide-based coloring reagents for the delivery of colorants (e.g. pigments, dyes, lakes, etc.) to a body surface have been developed to improve the durability of these compositions (Huang et al., U.S. Pat. No. 7,220,405 and U.S. Patent Application Publication No. 2005/0226839). The peptide-based colorants are prepared by coupling a specific peptide sequence that has a high binding affinity to a body surface with a coloring agent. The peptide portion binds to the body surface, thereby attaching the coloring agent to the body surface. Peptides with a high binding affinity for various body surfaces have been identified using phage display screening techniques (Huang et al., supra; Estell et al. WO 01/79479; Murray et al., U.S. Patent Application Publication No. 2002/0098524; Janssen et al., U.S. Patent Application Publication No. 2003/0152976; and Janssen et al., in WO 04/048399). However, the use of peptide-based coloring reagents comprising an iron oxide-binding peptide is not described.

Co-pending and co-owned U.S. Patent Application Publication No. 2007/0065387 reports the use of polymer coated pigment particles in peptide-based diblock and triblock conjugates for use in personal care compositions. Peptides having specific affinity for a polymeric coating were described. However, peptides having an affinity for uncoated pigment particles (i.e., uncoated iron oxide pigment) were not reported.

Pigment-binding peptides and peptide-based reagents comprising pigment-binding peptides have been reported. Specifically, co-owned U.S. Pat. No. 7,285,264 describes peptides having affinity for carbon black, CROMOPHTAL® Yellow, SUNFAST® Magenta, or SUNFAST® Blue. Although various other pigments are described, no iron oxide-binding peptide sequences are disclosed.

Co-pending U.S. Patent Application Publication No. 2007/0022547 describes pigment-binding peptides for use as peptide-based dispersion agents. However, no iron oxide-binding peptide sequences are disclosed.

European Patent EP1275728 B1 to Nomoto et al. describes peptides having high affinity for carbon black, copper phthalocyanine, titanium dioxide, and silicon dioxide. However, peptides having a specific affinity for iron oxide particles were not reported.

Escherichia coli mutants expression mutant versions of a plasmid born lamB gene (encoding the external domain of the phage λ receptor) were reported to have the ability to adhere to iron oxide particles (Brown, S., PNAS USA, (1992) 89:8651-8655). However, binding selectivity between the various metal oxides (i.e., Fe₂O₃, Fe₃O₄, mixed Fe₂O₃/Fe₃O₄, and Cr₂O₃) tested was limited. The reported interaction was not measured using purified peptide nor was the relative binding strength measured.

Whaley et al. (Nature 405:626-627 (2000)) describes several peptides that bind to metals and metal oxides used in the semiconductor industry, such as gallium arsenide and silicon. No specific iron oxide binding peptides are reported.

Sarikaya et al. (Nat. Mater. (2003) 2:577-585) provides a comprehensive review of biomimetic nanostructures that can be achieved using peptides selected against various inorganic surfaces, including SiO₂, CaCO₃, and Fe₂O₃. However, only a single peptide sequence is described that binds to Fe₂O₃.

Naik et al. describes in WO2003078451 (corresponding to U.S. Published Patent Application No. 2006/0035223) and in U.S. Published Patent Application No. 2006/0172282 several iron oxide-binding peptides identified by phage display. However, Naik et al. does not describe shampoo-resistant iron oxide-binding peptides nor does Naik et al. describe use of iron oxide binding peptides in peptide-based reagents for personal care.

In view of the above, a need exists to identify additional iron oxide-based pigment-binding peptides for use in peptide-based reagents for coloring body surfaces such as hair, skin, nails, and teeth. In a preferred embodiment, the iron oxide-based pigment-binding peptides are those capable of binding to the surface of an iron oxide-based pigment under highly stringent conditions, such as shampooing.

Applicants have addressed the stated need by identifying peptide sequences that bind with high affinity to iron oxide-based pigment particles. One or more of the present peptides can be coupled with one or more body surface-binding peptides to provide peptide-based reagents that may be used in combination with an iron oxide pigment in cosmetic applications to color body surfaces.

### SUMMARY OF THE INVENTION

The invention provides peptide-based reagents comprising at least one body surface-binding peptide and at least one of the present iron oxide-based pigment-binding peptides. These peptide-based reagents may be used in conjunction with an iron oxide-based pigment to color body surfaces, such as hair, skin, nails, and teeth. The body surface-binding peptide binds strongly to the body surface and the iron oxide-based pigment-binding peptide binds to the iron oxide pigment, thereby attaching the pigment to the body surface.

In one embodiment, a peptide-based reagent is provided selected from the group consisting of:

a) a peptide-based reagent having the general structure:

[(BSBP)ₘ-(IOBP)ₙ]ₓ; and

b) a peptide-based reagent having the general structure:

[[(BSBP)ₘ-S_{q}]ₓ-[(IOBP)ₙ-Sᵣ]_{z}]_{y};

wherein
i) BSBP is a body surface-binding peptide;
ii) IOBP is an iron oxide-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38;
iii) S is a spacer;
iv) m, n, x and z independently range from 1 to about 10;
v) y is from 1 to 5; and
vi) q an r are each independently 0 or 1, provided that both r and q may not be 0.

In another embodiment, a method of coloring a body surface with the peptide-based reagent is also provided comprising:
a) providing at least one iron oxide-based pigment;
b) providing a composition comprising at least one of the present peptide-based reagents; and
c) applying said at least one iron oxide-based pigment of (a) with the composition of (b) to a body surface for a time sufficient for the peptide-based reagent to bind to the iron oxide-based pigment and the body surface.

In another embodiment, the invention provides an iron oxide-binding peptide (IOBP) having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38.

In another embodiment, a personal care composition is provided comprising at least one of the present iron oxide-binding peptides or at least one of the present peptide-based reagents, and at least one iron oxide-based pigment.

### BRIEF DESCRIPTION OF FIGURES

The various embodiments of the invention can be more fully understood from the following figures, which form a part of this application.

FIG. 1 is a plasmid map of plasmid pLD001.

FIG. 2 is a plasmid map of plasmid pLD1475.

### BRIEF DESCRIPTION OF THE BIOLOGICAL SEQUENCES

The invention can be more fully understood from the following detailed description and the accompanying sequence descriptions, which form a part of this application.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOS: 1-38 are the amino acid sequences of the present iron oxide-binding peptides.

SEQ ID NO: 39 is the nucleic acid sequence of an oligonucleotide primer used to sequence phage DNA.

SEQ ID NO: 40 is the amino acid sequence of hair-binding peptide HP2.

SEQ ID NO: 41 is the amino acid sequence of hair-binding peptide Gray3.

SEQ ID NO: 42 is the amino acid sequence of the peptide linker TonB.

SEQ ID NO: 43 is the amino acid sequence of the hair-binding domain HP2-TonB-Gray3.

SEQ ID NO: 44 is the amino acid sequence of a peptide bridge used in the construction of peptide-based reagent HC353.

SEQ ID NO: 45 is the amino acid sequence of a peptide linker.

SEQ ID NO: 46 is the amino acid sequence of the peptide-based reagent HC353 comprising a hair-binding hand and a pigment-binding hand comprising two copies of the iron oxide-based pigment-binding peptide Rfe1.

SEQ ID NO: 47 is the nucleic acid sequence encoding the peptide reagent HC353.

SEQ ID NO: 48 is the nucleic acid sequence of plasmid pLD001.

SEQ ID NO: 49 is the amino acid sequence of solubility tag KSI(C4)E.

SEQ ID NO: 50 is the nucleic acid sequence of expression plasmid pLD1475.

SEQ ID NOs: 51-175 are the amino acid sequences of hair-binding peptides.

SEQ ID NOs: 171-223 are the amino acid sequences of skin-binding peptides.

SEQ ID NOs: 224-225 are the amino acid sequences of nail-binding peptides.

SEQ ID NOs: 226-265 are amino acid sequences of tooth-binding peptides.

SEQ ID NO: 266 is the amino acid sequence of the Caspase 3 cleavage site.

SEQ ID NOs:267-269 are the amino acid sequences of various peptide spacers.

### DETAILED DESCRIPTION

Iron oxide-binding peptides are provided having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38. The iron oxide-binding peptides were selected by phage display biopanning using an iron oxide-based pigment. As such, the iron oxide-binding peptides are alternatively referred to herein as “iron oxide-based pigment-binding peptides”.

The iron oxide-based pigment-binding peptides may be used to prepare peptide-based reagents for coupling at least one iron oxide-based pigment to a body surface for use in personal care compositions. In one embodiment, the personal care compositions are suitable for use in cosmetic coloring applications.

The following definitions are used herein and should be referred to for interpretation of the claims and the specification.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

The term “body surface” refers to any surface of the human body that may serve as a substrate for the binding of a peptide-based reagent and an iron oxide-based pigment particle. Typical body surfaces include but are not limited to hair, skin, nails, teeth, and tissues of the oral cavity, such as gums.

As used herein, “BSBP” refers to a body surface-binding peptide selected from the group consisting of hair-binding peptides, skin-binding peptides, nail-binding peptides, tooth-binding peptides, and peptides that have a specific affinity for oral cavity tissues, such as the gums. A body surface-binding peptide is a peptide that binds with high affinity to at least one body surface. Each target surface-binding peptide (such as a body-surface-binding peptide or one of the present iron oxide-binding peptides) will be referred to herein as a binding “finger”. Linking together multiple “fingers” forms a binding “domain” (also referred to herein as a binding “hand”). The body surface-binding peptide may be selected from the group consisting of hair-binding peptides, skin-binding peptides, nail-binding peptides, tooth-binding peptides, and oral cavity surface-binding peptides. In a preferred embodiment, the body surface-binding peptide is a hair-binding peptide, a skin-binding peptide or a tooth-binding peptide.

As used herein, “IOBP” refers to a peptide having affinity for iron oxide and is referred to herein as an “iron oxide-binding peptide” or an “iron oxide-based pigment-binding peptide”. The present peptides having affinity for iron oxide were identified by biopanning (using phage display) based on their affinity for iron oxide-based pigment(s).

As used herein, “S” means “spacer” or “linker”. In one embodiment, the spacer may be a peptide linker. In another embodiment, the spacer may be a peptide bridge.

As used herein, the term “peptide linker” refers to a peptide ranging in size from 1 to 60 amino acids in length, preferably 3 to 50 amino acids in length, which is used to link together two target surface-binding peptides (“fingers”) to form a binding domain (“hand”). In one embodiment, the peptide linker, when not used in forming a binding domain, is not typically characterized as having a strong affinity for the target surface.

As used herein, the term “peptide bridge” refers to a peptide ranging in size from 1 to 60 amino acids in length that is used to link together two binding domains (“hands”) or to link together a single binding “hand” directly to a benefit agent. In one embodiment, the peptide bridge, when not used in coupling together two or more binding domains, is not typically characterized as having a strong affinity for the target surface.

As used herein, the terms “iron oxide-based pigment” and “iron oxide pigment” will refer to a pigment particle comprised primarily of an iron oxide. Iron oxide pigments may vary in color (red, yellow, brown, and black tones) due to minor impurities and/or the size of the pigment particle. In one embodiment, the iron oxide pigment is a cosmetically acceptable iron oxide pigment. Cosmetically-acceptable iron oxide pigments are commercially available from various companies, such as Sensient Technologies Corp, Milwaukee, Wis. In one embodiment, the iron oxide is selected from the group consisting of ferric oxide (Fe₂O₃), ferrous ferric oxide (Fe₃O₄), and mixtures of Fe₂O₃ and Fe₃O₄. In one embodiment, the iron oxide is ferric oxide Fe₂O₃. In another embodiment, a portion of the iron oxide-based pigment may further comprise some silica.

As used herein, the term “hair” as used herein refers to human hair, eyebrows, and eyelashes. As used herein, the term “hair-binding peptide” (HBP) refers to a peptide that binds with strong affinity to hair. Hair binding peptides may include one or more hair binding domains. Examples of hair-binding peptides are provided as SEQ ID NOs: 40, 41, 51-175, and 225.

As used herein, the term “skin” as used herein refers to human skin, or substitutes for human skin, such as pig skin, VITRO-SKIN® (Innovative Measurement Solutions Inc., Milford, Conn.) and EPIDERM™ (MatTek Corporation, Ashland, Mass.). Skin, as used herein, will refer to a body surface generally comprising a layer of epithelial cells and may additionally comprise a layer of endothelial cells.

As used herein, the term “skin-binding peptide” (SBP) refers to peptides that bind with high affinity to skin. Examples of skin-binding peptides have also been reported (U.S. Pat. No. 7,309,482 to Buseman-Williams; WO 2004/000257 to Rothe et al.; and U.S. patent application Ser. No. 11/696380). Examples of skin-binding peptides are provided as SEQ ID NOs: 171-223.

As used herein, the term “nails” as used herein refers to human fingernails and toenails. As used herein, the term “nail-binding peptide” (NBP) refers to peptide sequences that bind with high affinity to nail. Examples of nail-binding peptides are provided as SEQ ID NOs: 224-225.

As used herein, the term “oral cavity surface-binding peptide” refers to peptides that bind with high affinity to surfaces such as teeth, gums, cheeks, tongue, or other surfaces in the oral cavity.

The term “tooth surface” will refer to both tooth enamel and tooth pellicle surfaces of mammalian teeth. In a preferred embodiment, the tooth surface will refer to both tooth enamel and tooth pellicle surfaces of human teeth. As such, both tooth enamel-binding peptides and tooth pellicle-binding peptides will be collectively referred to as tooth-binding peptides.

As used herein, the terms “pellicle” and “tooth pellicle” will refer to the thin film (typically about 1 to about 200 μm thick) derived from salivary glycoproteins which forms over the surface of the tooth crown.

As used herein, the terms “enamel” and “tooth enamel” will refer to the highly mineralized tissue which forms the outer layer of the tooth. The enamel layer is composed primarily of crystalline calcium phosphate (i.e., hydroxyapatite) along with water and some organic material.

As used herein, the term “tooth-binding peptide” (TBP) will refer to a peptide that binds with high affinity to tooth enamel or tooth pellicle. Examples of tooth-binding peptides having been disclosed in co-owed and co-pending U.S. Patent Application Publication No. 2008-0280810 and are provided as SEQ ID NOs: 226-265. Examples of tooth pellicle-binding peptides are provided as SEQ ID NOs: 226-245 and examples of tooth enamel-binding peptides are provided as SEQ ID NOs: 246-265. In one embodiment, the oral cavity surface-binding peptide is a peptide that binds with high affinity to tooth enamel and/or tooth pellicle.

The term “peptide” refers to two or more amino acids joined to each other by peptide bonds or modified peptide bonds.

The terms “coupling” and “coupled” as used herein refer to any chemical association and includes both covalent and non-covalent interactions. In one embodiment, coupling between the present peptides and peptide-based reagents and their respective surfaces is a non-covalent interaction.

The term “stringency” as it is applied to the selection of the body-surface-binding peptides, refers to the concentration of the eluting agent (such as a detergent) used to elute peptides from the body surface. Higher concentrations of the eluting agent provide more stringent conditions. The present iron oxide-binding peptides were selected under highly stringent conditions (i.e., peptides resistant to stringent washing conditions that include 0.5 wt % TWEEN® 20 and 30 wt % shampoo).

The term “MB₅₀” refers to the concentration of the binding peptide that gives a signal that is 50% of the maximum signal obtained in an ELISA-based binding assay (See Example 9 of U.S. Published Patent Application No. 2005-0226839). The MB₅₀ value provides an indication of the strength of the binding interaction or affinity of the components of the complex. Lower MB₅₀ values correlate with a stronger binding affinity between the peptide and the respective substrate.

The term “binding affinity” refers to the strength of the interaction of a binding peptide with its respective substrate. The binding affinity is defined herein in terms of the MB₅₀ value, determined in an ELISA-based binding assay. In one embodiment, “high affinity” or “strong affinity” is defined as an MB₅₀ value of 10⁻⁴ M or less, preferably 10⁻⁵M or less, even more preferably 10⁻⁶ M or less, and most preferably 10⁻⁷ M or less.

The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any naturally-occurring amino acid | Xaa | X |
| (or as defined herein) | | |

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Iron Oxide-Binding Peptides

Iron oxide-binding peptides as defined herein are peptide sequences that bind with high affinity to an iron oxide-based, such as an iron oxide-based pigment. In one embodiment, the iron oxide-based pigment is selected from the group consisting of ferric oxide (Fe₂O₃), ferrous ferric oxide (Fe₃O₄), and mixtures of Fe₂O₃ and Fe₃O₄. In a preferred embodiment, the iron oxide is Fe₂O₃. In one embodiment, the iron oxide-based pigment is a pigment particle comprising iron oxide. In another embodiment, the iron oxide-based pigment comprises iron oxide and some silica.

Peptides having an affinity for a target surface (i.e., target surface-binding peptides) may be selected using combinatorial methods that are well known in the art or may be empirically generated. The present iron oxide-based pigment binding peptides of the invention have a binding affinity for the iron oxide-based particle substrate, as measured by MB₅₀ values, of less than or equal to about 10⁻⁴ M, preferably less than or equal to about 10⁻⁵ M, more preferably less than or equal to about 10⁻⁶ M, more preferably less than or equal to about 10⁻⁷ M, even more preferably less than or equal to about 10⁻⁸ M, and even more preferably less than or equal to about 10⁻⁹ M.

The iron oxide-based pigment-binding peptides of the present invention are preferably combinatorially-generated and range in length from about 7 amino acids to about 60 amino acids, more preferably from about 7 amino acids to about 35 amino acids in length, and most preferably about 7 to about 20 amino acids in length. The iron oxide-based pigment-binding peptides of the present invention may be generated randomly and then selected against an iron oxide-based pigment. The generation of random libraries of peptides is well known and may be accomplished by a variety of techniques including, but not limited to bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate such biological peptide libraries are described in Dani, M., J. of Receptor & Signal Transduction Res., 21(4):447-468 (2001). Additionally, phage display libraries are available commercially from companies such as New England BioLabs (Beverly, Mass.). The disclosures of all United States Patents and published patent applications referred to in this paragraph are hereby incorporated by reference.

Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

More specifically, after a suitable library of peptides has been generated or purchased, the library is then contacted with an appropriate amount of the test substrate. The library of peptides is dissolved in a suitable solution for contacting the sample. The sample is typically suspended in solution or may be immobilized on a plate or bead. A preferred solution is a buffered aqueous saline solution containing a surfactant. A suitable solution is Tris-buffered saline (TBS) with 0.5% TWEEN® 20. The solution may additionally be agitated by any means in order to increase the mass transfer rate of the peptides to the target sample/surface, thereby shortening the time required to attain maximum binding.

Upon contact, a number of the randomly generated peptides will bind to the target surface to form a peptide-target surface complex, for example, peptide-iron oxide pigment. Unbound peptide may be removed by washing. After all unbound material is removed, peptides having varying degrees of binding affinities for the test surface may be fractionated by selected washings in buffers having varying stringencies. Increasing the stringency of the buffer used increases the required strength of the bond between the peptide and target surface in the peptide-target surface complex.

A number of substances may be used to vary the stringency of the washing solution in the peptide selection process including, but not limited to acids (pH 1.5-3.0), bases (pH 10-12.5), salts of high concentrations such as MgCl₂ (3-5 M) and LiCl (5-10 M), ethylene glycol (25-50%), dioxane (5-20%), thiocyanate (1-5 M), guanidine (2-5 M), urea (2-8 M), and surfactants of various concentrations such as SDS (sodium dodecyl sulfate), DOC (sodium deoxycholate), Nonidet P-40, Triton X-100, shampoo (useful when selecting peptides for use in personal care compositions, such as a commercial shampoo formulation), TWEEN® 20, wherein TWEEN® 20 is more typical. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for target surface substrates may be eluted by repeating the selection process using buffers with increasing stringencies. The eluted peptides can be identified and sequenced by any means known in the art.

As many of the peptide-based reagents will be used in personal care products comprising significant amounts of surfactants/detergents (such as a shampoo or a skin cleanser), the stringency of the washing steps may be increased to select only those peptides having the highest binding affinity. In one embodiment, the washing conditions will include at least 1 wt % shampoo, preferably at least 5 wt %, even more preferably at least 10 wt %, even more preferably at least 20 wt %, and most preferably at least 30 wt % shampoo. In one embodiment, peptides that are resistant to washing conditions that includes a shampoo will be referred to herein as “shampoo resistant”. In one embodiment, preferred peptides are those that are resistant to washing conditions that include at least 30 wt % shampoo (referred to herein as “shampoo-resistant iron oxide-based pigment-binding peptides”).

The present iron oxide-based pigment-binding peptides were identified using the methods described herein. The present iron oxide-based pigment-binding peptides comprise an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38.

### Body Surfaces

Body surfaces are any surface on the human body that will serve as a substrate for a binding peptide. Typical body surfaces include, but are not limited to hair, skin, nail, teeth, gums, and the tissues of the oral cavity. In many cases the body surfaces of the invention will be exposed to air, however in some instances, the oral cavity for example, the surfaces will be internal. Accordingly, body surfaces may include layers of both epithelial and well as endothelial cells.

Samples of body surfaces are available from a variety of sources. For example, human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated for example using hydrogen peroxide to obtain bleached hair. Human skin samples may be obtained from cadavers or in vitro human skin cultures. Additionally, pig skin, available from butcher shops and supermarkets, VITRO-SKIN®, available from IMS Inc. (Milford, Conn.), and EPIDERM™, available from MatTek Corp. (Ashland, Mass.), are good substitutes for human skin. Human fingernails and toenails may be obtained from volunteers. Extracted mammalian teeth, such as bovine and/or human teeth are commercially available. Extracted human teeth may also be obtained from dental offices. Additionally, hydroxyapatite, available in many forms, for example, from Berkeley Advanced Biomaterials, Inc. (San Leandro, Calif.), may be used (once coated with salivary glycoproteins to form an acquired pellicle) as a model for studying teeth-binding peptides (see U.S. Patent Application Publication No. 2008-0280810).

### Body Surface-Binding Peptides

Body surface-binding peptides as defined herein are peptide sequences that specifically bind with strong affinity to a respective target body surface including, but not limited to hair, nails, skin, teeth, and tissues of the oral cavity (such as gums). In one embodiment, the body surface is a hair, skin, nail, or tooth surface. In one embodiment, the body surface-binding peptide are selected from the group consisting of hair-binding peptides, skin-binding peptides, nail-binding peptides, and tooth-binding peptides.

Phage display has been used to identify various body surface-binding peptides. For example, peptides having an affinity for a body surface have been described in U.S. Pat. Nos. 7,220,405 and 7,285,264; U.S. Patent Application Publications Nos. US 2005-0226839, US 2005-0249682, US 2006-0073111, US 2006-0199206, US 2007-0065387, US 2007-0067924, US 2007-0196305, US 2007-0110686, US 2008-0280810, and US 2008-0175798; and PCT Patent Application Publication No. WO2004048399.

Alternatively, hair-binding and skin-binding peptide sequences may be generated empirically by designing peptides that comprise positively charged amino acids, which can bind to hair and skin via electrostatic interaction, as described by Rothe et al. (U.S. Pat. No. 7,341,604). The empirically generated hair and skin-binding peptides have between about 4 amino acids to about 50 amino acids, preferably from about 4 to about 25 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically generated hair-binding and skin peptides may include, but are not limited to, SEQ ID NOs: 171-175.

### Production of Binding Peptides

The iron oxide-based pigment-binding peptides, as well as any suitable body surface-binding peptides, may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, target surface-binding peptides as well as single chain peptide-based reagents (particularly when the entire diblock or triblock peptide-based coloring reagent is produced as a single amino acid chain) may be prepared using recombinant DNA and molecular cloning techniques. Genes encoding the peptides may be produced in heterologous host cells, particularly in the cells of microbial hosts.

Preferred heterologous host cells for expression of the binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides. Such vectors include, but are not limited to, chromosomal, episomal and virus-derived vectors, such as vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence, as well as an appropriate promoter or control sequence, may be employed to transform an appropriate host to permit the host to express the peptide of interest. Cell-free translation systems can also be employed to produce such peptides using RNAs derived from the DNA constructs. Optionally it may be desired to produce the gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are known in the art (see for example EP 546049 and WO 93/24631). The secretion signal DNA or facilitator may be located between the expression-controlling DNA and the gene or gene fragment, and in the same reading frame with the latter.

### Peptide-Based Reagents

The peptide-based reagents (diblock and/or triblock) are single chain peptides formed by coupling at least one body surface-binding peptide to at least one of the present iron oxide-binding peptides, either directly or through a molecular spacer. The part of the reagent comprising at least one body surface-binding peptide has affinity for the body surface, while the part of the reagent comprising at least one of present iron oxide-based pigment-binding peptides has strong affinity for an iron oxide-based pigment, thereby coupling the iron oxide-based pigment to the body surface.

In one embodiment, the peptide-based reagent comprising 1) at least one body surface-binding domain (also referred to herein as a “hand”) comprising two or more body surface-binding peptides (referred to herein as peptide “fingers”) optionally linked together by a peptide linker and 2) at least one of the present iron oxide-based pigment-binding peptides. In another embodiment, the peptide-based reagent comprises 1) at least body surface binding hand and 2) at least one iron oxide-based pigment-binding domain, separated optionally by a peptide bridge; wherein the inclusion of a peptide bridge is preferred. An example of a peptide-based reagent comprising at least one body surface-binding hand and at least one iron oxide-based pigment binding domain is provided as SEQ ID NO: 46.

The coupling interaction between the peptide-based reagent and the iron oxide-based pigment may be a covalent bond or a non-covalent interaction, such as hydrogen bonding, electrostatic interaction, hydrophobic interaction, or Van der Waals interaction. In the case of a non-covalent interaction, coupling of the peptide-based reagent to the iron oxide-based pigment may occur by simply mixing said at least one peptide-based reagent and at least one iron oxide-based pigment. The unbound materials may be separated from the resulting peptide-based reagent using methods known in the art, for example, gel permeation chromatography.

The peptide-based reagent may also be covalently attached to at least one iron oxide-binding peptide, either directly or through a spacer. Any known peptide or protein conjugation chemistry may be used to form the peptide-based reagents of the invention.

In one embodiment, the surface of the iron oxide-based pigment may be modified to enable covalent coupling of the peptide-based reagent to the surface of the iron oxide-based pigment. Conjugation chemistries are well-known in the art (see for example, Hermanson, Bioconjugate Techniques, Academic Press, New York, N.Y. (2008)). Suitable coupling agents may include, but are not limited to, carbodiimide coupling agents, diacid chlorides, diisocyanates and other difunctional coupling reagents that are reactive toward terminal amine and/or carboxylic acid groups. The preferred coupling agents are carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups. Additionally, it may be necessary to protect reactive amine or carboxylic acid groups on the peptides to produce the desired structure for the peptide-based reagent. The use of protecting groups for amino acids, such as t-butyloxycarbonyl (t-Boc), are well known in the art (see for example Stewart et al., supra; Bodanszky, supra; and Pennington et al., supra).

It may also be desirable to couple the body surface-binding peptide to the iron oxide-binding peptide via a spacer/linker to form a triblock peptide reagent. The spacer serves to separate the binding peptide sequences to ensure that the binding affinity of the individual peptides is not adversely affected by the coupling. The spacer may also provide other desirable properties such as hydrophilicity, hydrophobicity, or a means for cleaving the peptide sequences to facilitate removal of the coloring agent.

The “spacer” may also be any of a variety of molecules, such as alkyl chains, phenyl compounds, ethylene glycol, amides, esters and the like. In one embodiment, the organic spacers are hydrophilic and have a chain length from 1 to about 100 atoms, more preferably, from 2 to about 30 atoms. Examples of spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the body surface-binding and iron oxide-based pigment-binding peptide sequences using any of the coupling chemistries described above. In order to facilitate incorporation of the spacer, a bifunctional cross-linking agent that contains a spacer and reactive groups at both ends for coupling to the peptides may be used. Suitable bifunctional cross-linking agents are well known in the art and may include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents may include, but are not limited to compounds having the following structure:

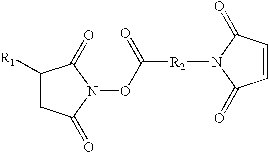

where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional cross-linking agent is 3-maleimidopropionic acid N-hydroxysuccinimide ester. The N-hydroxysuccinimide ester group of these reagents reacts with amine groups on one peptide, while the maleimide group reacts with thiol groups present on the other peptide. A thiol group may be incorporated into the peptide by adding at least one cysteine group to at least one end of the binding peptide sequence (i.e., the C-terminus and/or or N-terminus). Several spacer amino acid residues, such as glycine, may be incorporated between the binding peptide sequence and the terminal cysteine to separate the reacting thiol group from the binding sequence. Moreover, at least one lysine residue may be added to at least one end of the binding peptide sequence to provide an amine group for coupling.

Additionally, the “spacer” may be a peptide spacer (optionally referred to herein as a peptide “bridge” [when connecting two different binding domains or “hands”] or a peptide “linker” [when connecting two body- or pigment-binding peptides (“fingers”) to form a binding domain (a binding “hand”)]. The peptide spacer may range in size from 1 to 60 amino acids in length. In one embodiment, the peptide linker ranges from 3 amino acids to about 50 amino acids in length and has limit flexibility (i.e., a “rigid peptide linker”; see U.S. Provisional Patent Application No. 61/138,633). An example of a rigid peptide linker is provided as SEQ ID NO: 42 (the “TonB” linker). When the peptide spacer is used as a peptide bridge, the peptide bridge may range from about 1 amino acid to about 60 amino acids in length. In addition, the peptide spacer may contain a specific enzyme cleavage site, such as the protease Caspase 3 cleavage site, provided herein as SEQ ID NO: 266, which may be used for enzymatic removal of the pigment from the hair.

The spacer may be a peptide linker and may range in length from 1 amino acid to about 60 amino acids, preferably from 6 to about 60, and more preferably 3 to about 50 amino acids in length. Examples of suitable peptide linkers/spacers may include, but are not limited to, the sequences given by SEQ ID NOs: 42, 44, 45, and 267-269. These peptide spacers may be linked to the binding peptide sequences by any method known in the art. For example, the entire peptide-based reagent may be prepared using the standard peptide synthesis methods described, supra. In addition, the binding peptides and peptide spacer region may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid groups on the peptides, as described above. Alternatively, the entire triblock peptide-based reagent may be prepared using the recombinant DNA and molecular cloning techniques described supra. The spacer may also be a combination of a peptide spacer and an organic spacer molecule.

It may also be desirable to have multiple copies of the body surface-binding peptide and the iron oxide-binding peptide coupled together to enhance the binding affinity between the peptide-based reagent. Multiple copies of the same body surface-binding peptide and iron oxide-binding peptide or a combination of different body surface-binding peptides and iron oxide-binding peptides may be used, so long as the composition comprises at least one of the present iron oxide -binding peptides. The multi-copy peptide-based reagents may comprise various spacers as described above.

In one embodiment, the peptide-based reagent is composition comprising at least one body surface-binding peptide (BSBP) and at least one of the present iron oxide-binding peptides (IOBP), having the general structure [(BSBP)ₘ-(IOBP)ₙ]ₓ, where n and m independently range from 1 to about 10, preferably from 1 to about 5, and x may be 1 to about 10.

In another embodiment, the peptide-based reagent comprises a molecular spacer (S) separating the body surface-binding peptide from the iron oxide-binding peptide, as described above. Multiple copies of the body surface-binding peptide and the iron oxide-binding peptide may also be used and the multiple copies of the body surface-binding peptide and the iron oxide-binding peptide may be separated from themselves and from each other by molecular spacers. In this embodiment, the peptide-based reagent is a composition comprising at least one body surface-binding peptide, at least one spacer, and at least one of the present iron oxide-binding peptides, having the general structure [[(BSBP)ₘ-S_{q}]ₓ-[(IOBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z range from 1 to about 3.

In another embodiment, the body surface-binding peptide is a hair-binding peptide and the peptide-based reagent is a composition comprising at least one hair-binding peptide (HBP) and at least one of the present iron oxide-binding peptides (IOBP), having the general structure [(HBP)ₘ-(IOBP)ₙ]ₓ where n and m independently range from 1 to about 10, preferably from 1 to about 5, and x may be 1 to about 10.

In another embodiment, the body surface-binding peptide is a hair-binding peptide and the peptide-based reagent is a composition comprising at least one hair-binding peptide (HBP), at least one spacer (S), and at least one of the present iron oxide-binding peptides (IOBP), having the general structure [[(HBP)ₘ-S_{q}]ₓ-[(IOBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a skin-binding peptide and the peptide-based reagent is a composition comprising at least one skin-binding peptide (SBP) and at least one of the present iron oxide-binding peptides (IOBP), having the general structure [(SBP)ₘ-(IOBP)ₙ]ₓ, where n and m independently range from 1 to about 10, preferably from 1 to about 5, and x may be 1 to about 10.

In another embodiment, the body surface-binding peptide is a skin-binding peptide and the peptide-based reagent is a composition comprising at least one skin-binding peptide (SBP), at least one spacer (S), and at least one of the present iron oxide-binding peptides (IOBP), having the general structure [[(SBP)ₘ-S_{q}]ₓ-[(IOBP)ₙ-Sᵣ]_{z}]_{y}, where m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a nail-binding peptide and the peptide-based reagent is a composition comprising at least one nail-binding peptide (NBP) and at least one of the present iron oxide-binding peptides (IOPB), having the general structure [(NBP)ₘ-(IOBP)ₙ]ₓ where n and m independently range from 1 to about 10, preferably from 1 to about 5, and x may be 1 to about 10.

In another embodiment, the body surface-binding peptide is a nail-binding peptide and the peptide-based reagent is a composition comprising at least one nail-binding peptide (NBP), at least one spacer (S), and at least one of the present iron oxide-binding peptides (IOBP), having the general structure [[(NBP)ₘ-S_{q}]ₓ-[(IOBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a tooth-binding peptide and the peptide-based reagent is a composition comprising at least one tooth-binding peptide (TBP) and at least one of the present iron oxide-binding peptides (IOBP), having the general structure [(TBP)ₘ-(IOBP)ₙ]ₓ where n and m independently range from 1 to about 10, preferably from 1 to about 5, and x may be 1 to about 10.

In another embodiment, the body surface-binding peptide is a tooth-binding peptide and the peptide-based reagent is a composition comprising at least one tooth-binding peptide (TBP), at least one spacer (S), and at least one of the present iron oxide-binding peptides (IOBP), having the general structure [[(TBP)ₘ-S_{q}]ₓ-[(IOBP)ₙ-Sᵣ]_{z}]_{y}, where n, m, x, and z independently range from 1 to about 10, y is from 1 to about 5, and where q and r are each independently 0 or 1, provided that both q and r are not 0. In a further embodiment, m and n independently range from 1 to about 5, and x and z independently range from 1 to about 3.

It should be understood that as used herein BSBP, HBP, SBP, NBP, and TBP are generic designations and are not meant to refer to a single body surface-binding peptide, hair-binding peptide, skin-binding peptide, nail-binding peptide, or a tooth-binding peptide, respectively. Where m or n as used above is greater than 1, it is well within the scope of the invention to provide for the situation where a series of body surface-binding peptides of different sequences and iron oxide-binding peptides of different sequences may form a part of the composition. Additionally, S is a generic term and is not meant to refer to a single spacer. Where x and y, as used above for the triblock compositions, are greater than 1, it is well within the scope of the invention to provide for the situation where a series of different spacers may form a part of the composition. It should also be understood that these structures do not necessarily represent a covalent bond between the peptides and the optional molecular spacer. As described above, the coupling interaction between the peptides and the optional spacer may be either covalent or non-covalent. In a preferred embodiment, the peptide-based reagent is a linear, recombinantly produced peptide comprising at least one body surface-binding peptide, at least one of the present iron oxide-binding peptides, and optionally one or more peptide spacers.

### Personal Care Compositions

The present peptides and peptide-based reagents may be used in personal care compositions in conjunction with an iron oxide-based pigment to provide a benefit (such as color) to body surfaces, such as hair, skin, nails, and teeth. The peptide-based reagent may be present in the same composition as the iron oxide pigment, or the peptide-based reagent and the iron oxide pigment may be present in two different personal care compositions that are applied to the body surface in any order, as described below. Personal care compositions may include, but are not limited to, hair care/coloring compositions, skin care/coloring compositions, cosmetic compositions, nail care (such as nail polish) compositions, and oral care compositions.

Hair Care Compositions

The peptide-based reagent may be a component of a hair care composition; the peptide-based reagent comprising at least one hair-binding peptide and at least one of the present iron oxide-binding peptide. Hair care compositions are herein defined as compositions for the treatment of hair including, but not limited to, shampoos, conditioners, rinses, lotions, aerosols, gels, and mousses. An effective amount of the peptide-based reagent for use in hair care compositions is a concentration of about 0.01% to about 10%, preferably about 0.01% to about 5% by weight relative to the total weight of the composition. This proportion may vary as a function of the type of hair care composition. Additionally, the hair care composition may further comprise at least one pigment in addition to an iron oxide-based pigment. The concentration of the peptide-based reagent in relation to the concentration of the iron oxide-based pigment may need to be optimized for best results. Additionally, a mixture of different peptide-based reagents having an affinity for one or more additional pigments may be used in the composition to obtain the desired color. The peptide-based reagents in the mixture may be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents may be about 0.01% to about 10% by weight relative to the total weight of the composition.

The composition may further comprise a cosmetically-acceptable medium for hair care compositions, non-limiting examples of which are described by Philippe et al. in U.S. Pat. No. 6,280,747, and by Omura et al. in U.S. Pat. No. 6,139,851 and Cannell et al. in U.S. Pat. No. 6,013,250. For example, the hair care compositions may be aqueous, alcoholic or aqueous-alcoholic solutions, the alcohol preferably being ethanol or isopropanol, in a proportion of from about 1 to about 75% by weight relative to the total weight for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents, anionic, nonionic or amphoteric polymers, and dyes.

Hair Coloring Compositions

In another embodiment, the peptide-based reagent is a component of a hair coloring composition and the peptide-based reagent comprises at least one hair binding peptide and at least one of the present iron oxide-binding peptides. Hair coloring compositions are herein defined as compositions for the coloring or dyeing of hair, which comprise one or more coloring agents. Coloring agents as herein defined are comprised of at least one iron oxide pigment and may further include any dye, additional pigment(s), and the like that may be used to change the color of a body surface, such as hair, skin, nails, or teeth. Hair coloring agents are well known in the art (see for example Green et al. supra, CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany).

An effective amount of a peptide-based reagent (comprising at least one of the present iron oxide-binding peptides) for use in a hair coloring composition is herein defined as about 0.01% to about 20% by weight relative to the total weight of the composition. Additionally, a mixture of different peptide-based reagents having an affinity for different pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 20% by weight relative to the total weight of the composition.

Components of a cosmetically-acceptable medium for hair coloring compositions are described by Dias et al., in U.S. Pat. No. 6,398,821 and by Deutz et al., in U.S. Pat. No. 6,129,770, both of which are incorporated herein by reference. For example, hair coloring compositions may contain sequestrants, stabilizers, thickeners, buffers, carriers, surfactants, solvents, antioxidants, polymers, and conditioners.

Skin Care Compositions

In another embodiment, the peptide-based reagent is a component of a skin care composition and the peptide-based reagent comprises at least one skin-binding peptide and at least one of the present iron oxide-binding peptides. Skin care compositions are herein defined as compositions for the treatment of skin including, but not limited to, skin care, skin cleansing, make-up, and anti-wrinkle products. An effective amount of the peptide-based reagent for use in a skin care composition is a concentration of about 0.01% to about 10%, preferably about 0.01% to about 5% by weight relative to the total weight of the composition. This proportion may vary as a function of the type of skin care composition. Additionally, a mixture of different peptide-based reagents having an affinity for different (additional) pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 10% by weight relative to the total weight of the composition. The skin care composition may further comprise (in addition to an iron oxide-based pigment) at least one additional pigment, suitable examples of which are given above. The concentration of the peptide-based reagent in relation to the concentration of the pigment may need to be optimized for best results.

The composition may further comprise a cosmetically acceptable medium for skin care compositions, examples of which are described by Philippe et al., supra. For example, the cosmetically acceptable medium may be an anhydrous composition containing a fatty substance in a proportion generally of from about 10 to about 90% by weight relative to the total weight of the composition, where the fatty phase contains at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

Skin Coloring Compositions

In another embodiment, the peptide-based reagent is a component of a skin coloring composition and the peptide-based reagent comprises at least one skin-binding peptide and at least one of the present iron oxide-binding peptides. The skin coloring composition comprises one or more coloring agents in addition to at least one iron oxide-based pigment. Any of the coloring agents described above may be used.

The skin coloring compositions may be any cosmetic or make-up product, including but not limited to foundations, blushes, lipsticks, lip liners, lip glosses, eyeshadows and eyeliners. These may be anhydrous make-up products comprising a cosmetically acceptable medium which contains a fatty substance, or they may be in the form of a stable dispersion such as a water-in-oil or oil-in-water emulsion, as described above. In these compositions, an effective amount of the peptide-based reagent is generally from about 0.01% to about 40% by weight relative to the total weight of the composition. Additionally, a mixture of different peptide-based reagents having an affinity for different pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 40% by weight relative to the total weight of the composition.

Cosmetic Compositions

In another embodiment, the peptide-based reagent is a component of a cosmetic composition and the peptide-based reagent comprises at least one body surface-binding peptide and at least one of the present iron oxide-binding peptides, and an iron oxide pigment.

Cosmetic compositions, as defined herein, are compositions that may be applied to the eyelashes or eyebrows including, but not limited to mascaras, and eyebrow pencils. These cosmetic compositions may comprise one or more coloring agents in addition to at least one iron oxide pigment. Any of the coloring agents described above may be used.

An effective amount of a peptide-based reagent for use in a cosmetic composition is herein defined as a proportion of from about 0.01% to about 20% by weight relative to the total weight of the composition. Additionally, a mixture of different peptide-based reagents having affinity for different pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 20% by weight relative to the total weight of the composition.

Cosmetic compositions may be anhydrous make-up products comprising a cosmetically acceptable medium which contains a fatty substance in a proportion generally of from about 10 to about 90% by weight relative to the total weight of the composition, where the fatty phase containing at least one liquid, solid or semi-solid fatty substance, as described above. The fatty substance includes, but is not limited to, oils, waxes, gums, and so-called pasty fatty substances. Alternatively, these compositions may be in the form of a stable dispersion such as a water-in-oil or oil-in-water emulsion, as described above.

Nail Polish Compositions

In another embodiment, the peptide-based reagent is a component of a nail polish composition and the peptide-based reagent comprises at least one nail-binding peptide and at least one of the present iron oxide-binding peptides.

The nail polish compositions are used for coloring fingernails and toenails. The present nail polish compositions comprise at least one peptide-based coloring reagents and at least one iron oxide pigment. The nail polish compositions may contain one or more additional coloring agents. Any of the coloring agents described above may be used.

An effective amount of a peptide-based reagent for use in a nail polish composition is herein defined as a proportion of from about 0.01% to about 20% by weight relative to the total weight of the composition. Additionally, a mixture of different peptide-based reagents having affinity for different pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 20% by weight relative to the total weight of the composition.

Components of a cosmetically acceptable medium for nail polish compositions are described by Philippe et al., supra. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

Oral Care Compositions

In another embodiment, the peptide-based reagent is a component of an oral care composition and the peptide-based reagent comprises at least one tooth-binding peptide and at least one of the present iron oxide-binding peptides. Typically, oral care compositions comprise at least one white colorant and are used to whiten teeth. Suitable white colorants which may be used in the oral care composition include, but are not limited to, white pigments such as titanium dioxide and titanium dioxide nanoparticles; and white minerals such as hydroxyapatite, and Zircon (zirconium silicate). However, it may be desirable to further include at least one iron oxide pigment to an oral care composition even though iron oxides typically are not used to whiten teeth. In one embodiment, the peptide-based coloring reagent may be used to detect the presence of a particular surface on teeth (e.g., a diagnostic application). For example, the peptide-based coloring reagent may be used to detect the presence of a pellicle coating on teeth immediately after an abrasive cleaning/polishing procedure (such as a dental office cleaning/polishing procedure).

The oral care compositions of the invention may be in the form of powder, paste, gel, liquid, ointment, or tablet. Exemplary oral care compositions include, but are not limited to toothpaste, dental cream, gel or tooth powder, mouth wash, breath freshener, and dental floss. The oral care compositions comprise an effective amount of the peptide-based reagent of the invention in an orally acceptable carrier medium. An effective amount of a peptide-based reagent for use in an oral care composition may vary depending on the type of product. Typically, the effective amount of the peptide-based reagent is a proportion from about 0.01% to about 90% by weight relative to the total weight of the composition. Additionally, a mixture of different peptide-based reagents having affinity for different pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.001% to about 90% by weight relative to the total weight of the composition.

Examples of components suitable for use in an orally-acceptable carrier medium are described by White et al. in U.S. Pat. No. 6,740,311; Lawler et al. in U.S. Pat. No. 6,706,256; and Fuglsang et al. in U.S. Pat. No. 6,264,925; all of which are incorporated herein by reference. For example, the oral care composition may comprise one or more of the following: abrasives, surfactants, chelating agents, fluoride sources, thickening agents, buffering agents, solvents, humectants, carriers, bulking agents, and oral benefit agents, such as enzymes, anti-plaque agents, anti-staining agents, anti-microbial agents, anti-caries agents, flavoring agents, coolants, and salivating agents.

### Methods for Coloring a Body Surface

The peptide-based reagents of the invention may be used in conjunction with iron oxide pigment to color body surfaces, such as hair, skin, nails, and teeth. The body surface-binding peptide block of the peptide-based agent has an affinity for the body surface, while the iron oxide-binding peptide block has an affinity for an iron oxide-based pigment. The peptide-based reagent may be present in the same composition as the iron oxide pigment, or the peptide-based reagent and the iron oxide pigment may be present in two different compositions. In one embodiment, a personal care composition comprising at least one peptide-based agent and an iron oxide pigment is applied to a body surface for a time sufficient for the peptide-based agent, which is non-covalently coupled to the iron oxide pigment via the iron oxide-binding peptide block, to bind to the body surface. In another embodiment, at least one iron oxide pigment is applied to a body surface prior to the application of a composition comprising at least one peptide-based reagent. In another embodiment, a composition comprising at least one peptide-based reagent is applied to the body surface prior to the application of the iron oxide-based pigment. In another embodiment, at least one iron oxide pigment and a composition comprising at least one peptide-based reagent are applied to the body surface concomitantly. Optionally, the composition comprising the peptide-based reagent may be reapplied to the body surface after the application of the iron oxide pigment and the initial application of the composition comprising the peptide-based reagent. Additionally, a composition comprising a polymeric sealant may be applied to the body surface after the application of the iron oxide pigment and the composition comprising a peptide-based reagent.

Methods for Coloring Hair

The peptide-based reagent may be used to attach an iron oxide-based pigment to the surface of the hair, thereby coloring the hair. The peptide-based reagent and the pigment may be applied to the hair from any suitable hair care composition, for example a hair colorant, a hair shampoo or a hair conditioner composition. These hair care compositions are well known in the art and suitable compositions are described above.

In one embodiment, an iron oxide-based pigment is applied to the hair for a time sufficient for the iron oxide-based pigment to bind to the hair, typically between about 5 seconds to about 60 minutes. Optionally, the hair may be rinsed to remove the iron oxide-based pigment that has not bound to the hair. Then, a composition comprising a peptide-based reagent is applied to the hair for a time sufficient for the reagent to bind to the hair and the iron oxide-based pigment, typically between about 5 seconds to about 60 minutes. The composition comprising the peptide-based reagent may be rinsed from the hair or left on the hair.

In another embodiment, a composition comprising a peptide-based body surface reagent is applied to the hair for a time sufficient for the hair-binding peptide block of the reagent to bind to the hair, typically between about 5 seconds to about 60 minutes. Optionally, the hair may be rinsed to remove the composition that has not bound to the hair. Then, an iron oxide pigment is applied to the hair for a time sufficient for the iron oxide pigment to bind to the iron oxide-binding block of the reagent, typically between about 5 seconds to about 60 minutes. The unbound iron oxide pigment may be rinsed from the hair or left on the hair.

In another embodiment, an iron oxide pigment and a composition comprising a peptide-based reagent are applied to the hair concomitantly for a time sufficient for the reagent to bind to hair and the iron oxide pigment, typically between about 5 seconds to about 60 minutes. Optionally, the hair may be rinsed to remove the unbound iron oxide pigment and the composition comprising a peptide-based reagent from the hair.

In another embodiment, an iron oxide pigment is provided as part of a composition comprising a peptide-based reagent, for example a hair coloring composition. The composition comprising the iron oxide pigment and the reagent is applied to the hair for a time sufficient for the reagent, which is coupled to the iron oxide pigment through the iron oxide-binding peptide block, to bind to the hair, typically between about 5 seconds to about 60 minutes. The composition comprising the iron oxide pigment and the reagent may be rinsed from the hair or left on the hair.

In any of the methods described above, the composition comprising a peptide-based reagent may be optionally reapplied to the hair after the application of the iron oxide pigment and the initial application of the composition comprising a peptide-based reagent in order to further enhance the durability of the colorant.

Additionally, in any of the methods described above, a composition comprising a polymeric sealant may be optionally applied to the hair after the application of the iron oxide pigment and the composition comprising a peptide-based reagent in order to further enhance the durability of the colorant. The composition comprising the polymeric sealant may be an aqueous solution or a hair care composition, such as a conditioner or rinse, comprising the polymeric sealant. Typically, the polymeric sealant is present in the composition at a concentration of about 0.25% to about 10% by weight relative to the total weight of the composition. Polymeric sealants are well know in the art of personal care products and include, but are not limited to, poly(allylamine), acrylates, acrylate copolymers, polyurethanes, carbomers, methicones, amodimethicones, polyethylenene glycol, beeswax, siloxanes, and the like. The choice of polymeric sealant depends on the particular pigment and the peptide-based reagent used. The optimum polymeric sealant may be readily determined by one skilled in the art using routine experimentation.

Methods for Coloring Skin

The peptide-based reagents of the invention may be used to attach an iron oxide pigment to the surface of the skin, thereby coloring the skin. The peptide-based reagent and the pigment may be applied to the skin from any suitable skin care composition, for example a skin colorant or skin conditioner composition. These skin care compositions are well known in the art and suitable compositions are described above.

In one embodiment, an iron oxide pigment is applied to the skin for a time sufficient for the iron oxide pigment to bind to the skin, typically between about 5 seconds to about 60 minutes. Optionally, the skin may be rinsed to remove the pigment that has not bound to the skin. Then, a composition comprising a peptide-based reagent is applied to the skin for a time sufficient for the reagent to bind to the skin and the iron oxide pigment, typically between about 5 seconds to about 60 minutes. The composition comprising the peptide-based reagent may be rinsed from the skin or left on the skin.

In another embodiment, a composition comprising a peptide-based reagent is applied to the skin for a time sufficient for the skin-binding peptide block of the reagent to bind to the skin, typically between about 5 seconds to about 60 minutes. Optionally, the skin may be rinsed to remove the composition that has not bound to the skin. Then, an iron oxide-based pigment is applied to the skin for a time sufficient for the iron oxide pigment to bind to the iron oxide-binding block of the reagent, typically between about 5 seconds to about 60 minutes. The unbound iron oxide pigment may be rinsed from the skin or left on the skin.

In another embodiment, an iron oxide pigment and a composition comprising a peptide-based reagent are applied to the skin concomitantly for a time sufficient for the reagent to bind to skin and the iron oxide pigment, typically between about 5 seconds to about 60 minutes. Optionally, the skin may be rinsed to remove the unbound iron oxide pigment and the composition comprising a peptide-based reagent from the skin.

In another embodiment, an iron oxide pigment is provided as part of the composition comprising a peptide-based reagent, for example a skin coloring composition. The composition comprising the iron oxide pigment and the reagent is applied to the skin for a time sufficient for the reagent, which is coupled to the iron oxide pigment through the iron oxide-binding block, to bind to the skin, typically between about 5 seconds to about 60 minutes. The composition comprising the iron oxide pigment and the reagent may be rinsed from the skin or left on the skin.

In any of the methods described above, the composition comprising a peptide-based reagent may be optionally reapplied to the skin after the application of the iron oxide pigment and the initial application of the composition comprising a peptide-based reagent in order to further enhance the durability of the colorant.

Additionally, in any of the methods described above, a composition comprising a polymeric sealant may be optionally applied to the skin after the application of the iron oxide pigment and the composition comprising a peptide-based reagent in order to further enhance the durability of the colorant. Any of the polymeric sealants described above for hair coloring may be used in the form of an aqueous solution or a skin care composition.

Methods for Coloring Nails, Eyebrows, Eyelashes, and Teeth

The methods described above for coloring hair and skin may also be applied to coloring finger nails and toenails, eyebrows, eyelashes, and teeth by applying the appropriate composition, specifically, a nail polish composition, a cosmetic composition, or an oral care composition, to the body surface of interest.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “g” means the gravitation constant, “rpm” means revolution(s) per minute, “pfu” means plaque forming unit(s), “BSA” means bovine serum albumin, “ELISA” means enzyme linked immunosorbent assay, “IPTG” means isopropyl β-D-thiogalactopyranoside, “A” means absorbance, “A₄₅₀” means the absorbance measured at a wavelength of 450 nm, “OD₆₀₀” means the optical density measured at 600 nanometers, “TBS” means Tris-buffered saline, “TBST-X” means Tris-buffered saline containing TWEEN® 20 where “X” is the weight percent of TWEEN® 20, “Xgal” means 5-bromo-4-chloro-3-indolyl-beta-D-galactopyranoside, “SEM” means standard error of the mean, “vol %” means volume percent, “wt %” means weight percent, “NMR” means nuclear magnetic resonance spectroscopy, “MALDI mass spectrometry” means matrix assisted, laser desorption ionization mass spectrometry, “atm” means atmosphere(s), “kPa” means kilopascal(s), “SLPM” means standard liter(s) per minute, “psi” means pound(s) per square inch, “RCF” means relative centrifugal field.

General Methods:

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma-Aldrich Chemical Company (St. Louis, Mo.), unless otherwise specified.

### Example 1

### Selection of Peptides Having Affinity for Iron Oxide-Based Pigments Using Standard Biopanning

The purpose of this example was to identify phage peptides that bind iron oxide-based particles using phage display-based biopanning.

Commercial iron oxide particles were purchased from Sensient Technologies Corp, Milwaukee, Wis. (Unipure Red LC381EM, “red” iron oxide). Permanent double-sided tape (SCOTCH®; 3M Corp., Minneapolis, Minn.) was dipped in the iron oxide powder until fully coated. The iron oxide-coated tape was rinsed in 200 mL of water for three times. The tape was then rinsed in 200 mL of water gently shaking for 2 hours. The coated tape was cut into ½ cm×1 cm strips. The strips then were incubated in SUPERBLOCK® blocking buffer (Pierce Chemical Company, Rockford, Ill.; Prod. #37535) for 1 hour at room temperature, followed by 3 washes with TBST (TBS in 0.5% TWEEN® 20). Libraries of phage containing random peptide inserts (10¹¹ pfu) from 7 to 20 amino acids were added to each tube. After 60 minutes of incubation at room temperature and shaking at 50 rpm, unbound phage were removed by aspirating the liquid out of each well followed by 6 washes with 1.0 mL TBS containing the detergent TWEEN® 20 (TBST, T-0.5%) and 30% of Neutrogena shampoo (NEUTROGENA® Clean Replenishing, Moisturizing Shampoo, Neutrogena Corporation, Los Angeles, Calif. 90045).

The particle samples were then transferred to a clean tube, and 200 μL of elution buffer consisting of 1 mg/mL BSA (bovine serum albumin) in 0.2 M glycine-HCl, pH 2.2, was added to each well and incubated for 10 min to elute the bound phages. Then, 32 μL of neutralization buffer consisting of 1 M Tris-HCl, pH 9.2, was added to each tube. The phage particles, which were in the elution buffer as well as on the particles, were amplified by incubating with diluted E. coli ER2738 cells, from an overnight culture diluted 1:100 in LB medium, at 37° C. for 4.5 h. After this time, the cell culture was centrifuged for 30 seconds and the upper 80% of the supernatant was transferred to a fresh tube, ⅙ volume of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then tittered according to the standard protocol. For the 2^{nd}, 3^{rd} and 4^{th} round of biopanning, more than 2×10¹¹ pfu of phage stock from the previous round was used. The biopanning process was repeated under the same conditions as described above.

After the 4^{th} round of biopanning, 95 random single phage plaque lysates were prepared following the manufacture's instructions (New England BioLabs) and the single stranded phage genomic DNA was purified using the QIAprep Spin M13 Kit (Qiagen, Valencia, Calif.) and sequenced at the DuPont Sequencing Facility using −96 gIII sequencing primer (5′-CCCTCATAGTTAGCGTAACG-3′; SEQ ID NO: 39). The displayed peptide is located immediately after the signal peptide of gene III. Based on the peptide sequences, 30 phage candidates showed significant enrichment were selected for further pellicle binding analysis. The Amino acid sequences of selected phage candidates were listed in Table 1.

TABLE 1
Amino Acid Sequences of Peptide Having
Affinity for Iron Oxide-Based Particles

| Phage | | |
|---|---|---|
| ID | Amino Acid Sequences | SEQ ID NO: |
| Rfe1 | WAPEKDHMQLMK | 1 |
| Rfe2 | WAPEKDYMQLMK | 2 |
| Rfe3 | CPLDTPTHKTKHEYKTRCRH | 3 |
| Rfe4 | DHDHPRLHKRQEKSEHLH | 4 |
| Rfe5 | DSHHNHHKQDSRPQHRKTPN | 5 |
| Rfe6 | EGGNAPHHKPHHRKH | 6 |
| Rfe7 | HDSHRPLTQHGHRHSHVP | 7 |
| Rfe8 | HDSNHCSHSTRRPNCART | 8 |
| Rfe9 | ATRVDNTPASNPPSL | 9 |
| Rfe10 | DGIKPFHLMTPTLAN | 10 |
| Rfe11 | DITPPGSTHHRKPHRHQH | 11 |
| Rfe12 | DNLWPQPLNVEDDRY | 12 |
| Rfe13 | ENEKHRHNTHEALHSHFK | 13 |
| Rfe14 | GAIWPASSALMTEHNPTDNH | 14 |
| Rfe15 | GDTNQDTVMWYYTVN | 15 |
| Rfe16 | HNGPYGMLSTGKIHF | 16 |
| Rfe17 | LDGGYRDTPDNYLKG | 17 |
| Rfe18 | LHTKTENSHTNMKTT | 18 |
| Rfe19 | NAQYDPPTLNKGAVRKAAST | 19 |
| Rfe20 | NGNNHTDIPNRSSYT | 20 |
| Rfe21 | QSTNHHHPHAKHPRVNTH | 21 |
| Rfe22 | SNNDYVGTYPATAIQ | 22 |
| Rfe23 | STQHNLHDRNIYFVS | 23 |
| Rfe24 | TANNKTPAGAPNAAVGLAQR | 24 |
| Rfe25 | TEPTRISNYRSIPND | 25 |
| Rfe26 | THNPREHARHHHHNEYKH | 26 |
| Rfe27 | THPPCWYETNCIVQE | 27 |
| Rfe28 | TTNPHKPASHHHDHRPALRH | 28 |
| Rfe29 | WLVADNATDGHSHQK | 29 |
| Rfe30 | YTDSMSDQTPEFAKY | 30 |

### Example 2

### Characterization of Selected Peptides for Iron Oxide Binding Activities

Enzyme-linked immunosorbent assay (ELISA) was used to evaluate the iron oxide particle-binding affinity of the biopanning selected peptide candidates (Example 1; biotinylated peptides ID: Rfe1 through Rfe8). The identified peptides were synthesized using standard solid-phase synthesis method as described in U.S. Pat. No. 7,585,495. All peptides were modified to contain a biotinylated lysine residue at the C-terminus of the amino acid binding sequence for detection purposes (Table 2).

The iron oxide particles were dispersed in water at 2.5 mg per mL. The dispersion was made by vortexing the mixture for 1 min, which gave an average particle size of approximately 0.5 μm in diameter. The particle dispersion (1 mL each) was then centrifuged for 2 min at 5000 rpm. The liquid supernatant was removed by aspirating it out of each tube. The tubes were then incubated in SUPERBLOCK® blocking buffer (Pierce Chemical Company, Rockford, Ill.; Prod. #37535) for 1 hour at room temperature, followed by 3 washes with TBST (TBS in 0.05% TWEEN® 20). Then tubes were rinsed 3 times with wash buffer consisting of TBST-0.05% using the same centrifugation and aspiration methods. Peptide binding buffer consisting of 20 μM biotinylated peptides in TBST and 1 mg/mL BSA was added to the particles and incubated for 1 hour at room temperature (˜22° C.), followed by 6 washes with TBST. Then, the streptavidin-alkaline phosphatase (AP) conjugate TMB (3,3′,5,5′-tetramethylbenzidine), obtained from Pierce Biotechnology (Item #34021; Rockford, Ill.) was added to each well at standard concentration and incubated for 1 h at room temperature, followed by 6 washes with TBST. After the last wash, all particles were transferred to new tubes and then the color development and the absorbance measurements were performed following the standard protocols. The resulting absorbance values, reported as the mean of at least three replicates, and the standard error of the mean (SEM) are given in Table 2.

The results demonstrate that all of the hair-binding peptides tested had a higher iron oxide-based particle-binding activity than the control samples.

TABLE 2
Peptide Having Affinity for Iron Oxide-based
Pigment-Binding Peptide Results

| Peptide | Amino Acid Sequence | OD at | |
|---|---|---|---|
| ID | (SEQ ID NO.) | 405 nm | SEM |
| Control | No peptide | 0.08 | 0.003 |
| Rfe1- | WAPEKDHMQLMKK-biotin | 0.163 | 0.014 |
| biotin | (SEQ ID NO: 31) | | |
| Rfe2- | WAPEKDYMQLMKK-biotin | 0.206 | 0.024 |
| biotin | (SEQ ID NO: 32) | | |
| Rfe3- | CPLDTPTHKTKHEYKTRCRHK- | 1 | 0.01 |
| biotin | biotin | | |
| | (SEQ ID NO: 33) | | |
| Rfe4- | DHDHPRLHKRQEKSEHLHK- | 0.865 | 0.019 |
| biotin | biotin | | |
| | (SEQ ID NO: 34) | | |
| Rfe5- | DSHHNHHKQDSRPQHRKTPNK- | 0.795 | 0.049 |
| biotin | biotin | | |
| | (SEQ ID NO: 35) | | |
| Rfe6- | EGGNAPHHKPHHRKHK-biotin | 0.503 | 0.026 |
| biotin | (SEQ ID NO: 36) | | |
| Rfe7- | HDSHRPLTQHGHRHSHVPK- | 0.329 | 0.012 |
| biotin | biotin | | |
| | (SEQ ID NO: 37) | | |
| Rfe8- | HDSNHCSHSTRRPNCARTK- | 0.973 | 0.104 |
| biotin | biotin | | |
| | (SEQ ID NO: 38) | | |

### Example 3

### Determination of the Binding Affinity of Iron Oxide-Based Pigment-Binding Peptides

The purpose of this Example is to demonstrate the affinity of the iron oxide-based particle binding peptides for the particle surface, measured as MB₅₀ values, using an ELISA assay.

Iron Oxide-binding peptides, Rfe4, Rfe5, Rfe6 and Rfe7 identified using the methods described in Example 1 or Example 2 were synthesized by Synpep Inc. (Dublin, Calif.). The peptides were biotinylated by adding biotin on to a C-terminal lysine residue added to the respective peptide.

### MB₅₀ Measurement of Iron Oxide-Binding Peptide:

The MB₅₀ measurements of biotinylated peptides binding to iron oxide were conducted using a 96-well plate format. Iron oxide-based particles were added to the wells. The wells containing the iron oxide-based pigment powders were blocked with blocking buffer (SUPERBLOCK® from Pierce Chemical Co., Rockford, Ill.) at room temperature (˜22° C.) for 1 h, followed by six washes with TBST-0.5%, 2 min each, at room temperature. Various concentrations of biotinylated, binding peptide are added to each well, incubated for 1 hour at room temperature, and washed six times with TBST-0.5%, 2 min each, at room temperature. Then, streptavidin-horseradish peroxidase (HRP) conjugate (TMB) was added to each well (1.0 μg per well), and incubated for 1 h at room temperature. After the incubation, the wells were washed six times with TBST-0.5%, 2 min each at room temperature. Finally, the color development and the absorbance measurements were performed as described in Example 2.

The results were plotted as A₄₅₀ versus the concentration of peptide using GraphPad Prism 4.0 (GraphPad Software, Inc., San Diego, Calif.). The MB₅₀ values were calculated from Scatchard plots. The results are listed in Table 3.

TABLE 3

| Peptide | Amino Acid Sequence | |
|---|---|---|
| ID | (SEQ ID NO.) | MB₅₀ (M) |
| Rfe4 | DHDHPRLHKRQEKSEHLH-K- | 8 × 10⁻⁸ |
| | Biotin-NH₂ | |
| | (SEQ ID NO: 34) | |
| Rfe5 | DSHHNHHKQDSRPQHRKTPNK- | 2.8 × 10⁻⁷ |
| | Biotin-NH₂ | |
| | (SEQ ID NO: 35) | |
| Rfe6 | EGGNAPHHKPHHRKHK-Biotin- | 5.5 × 10⁻⁷ |
| | NH₂ | |
| | (SEQ ID NO: 36) | |
| Rfe7 | HDSHRPLTQHGHRHSHVPK- | 3 × 10⁻⁷ |
| | Biotin-NH₂ | |
| | (SEQ ID NO: 37) | |

### Example 4

### Construction of Peptide-Based Reagent Comprising Hair-Binding Domain and an Iron Oxide-Based Pigment Binding Domain

Hair-binding peptides designated HP2 (SEQ ID NO: 40) and Gray3 (SEQ ID NO: 41) were selected from random peptide libraries displayed fused to the pill protein of bacteriophage M13 for their ability to bind to human hair, using conventional phage display technology (Tim Clackson and Henry B. Lowman, Eds., Phaqe Display: A Practical Approach, Oxford University Press, New York, N.Y. (2004)). The iron oxide-based pigment binding peptide designated as “Rfe1” was selected for the preparation of the peptide-based reagent (SEQ ID NO: 1; Example 1).

The combination of hair-binding peptides HP2 (SEQ ID NO: 40) and Gray3 (SEQ ID NO: 41) and the linker joining them (TonB; SEQ ID NO: 42) were selected from a combinatorial library consisting of module combinations of the type [binding sequence-linker-binding sequence], using “monovalent” phage display technology.

The HP2-TonB-Gray3 (SEQ ID NO: 43) hair-binding hand was coupled via a peptide bridge (GSGGGGSP; SEQ ID NO: 44) to an iron oxide-based pigment-binding hand comprising two iron oxide-based pigment-binding peptides (Rfe1×2) linked together by a cationic peptide linker (GKGKGKGKGKGKGKGKGKGKG; SEQ ID NO: 45), to form peptide-based reagent “HC353”. The target surface-binding peptides are in bold. The rigid linker is italicized.

Formula for Peptide-Based Reagent HC353
PS-HP2-GP-TonB-PA-Gray3-GSGGGGSP-Rfe1-
GKGKGKGKGKGKGKGKGKGKG-Rfe1-GK
Corresponding Peptide Sequence for HC353

(SEQ ID NO: 46)

PSAQSQLPDKHSGLHERAPQRYGPEPEPEPEPIPEPPKEAPWIEKPKPKP
KPKPKPPAHDHKNQKETHQRHAAGSGGGGSPWAPEKDHMQLMKGKGKGKG
KGKGKGKGKGKGKGWAPEKDHMQLMKGK

### Construction of the DNA Coding Sequence

The DNA sequence (SEQ ID NO: 47) encoding the HC353 peptide-based reagent was assembled by DNA2.0 Inc. (Menlo Park, Calif.) using conventional chemical synthesis of DNA and assembly from oligonucleotides by annealing and ligation. Candidate sequences were cloned into a vector and verified by DNA sequencing by DNA2.0.

Recloning into Expression Vector pLD001

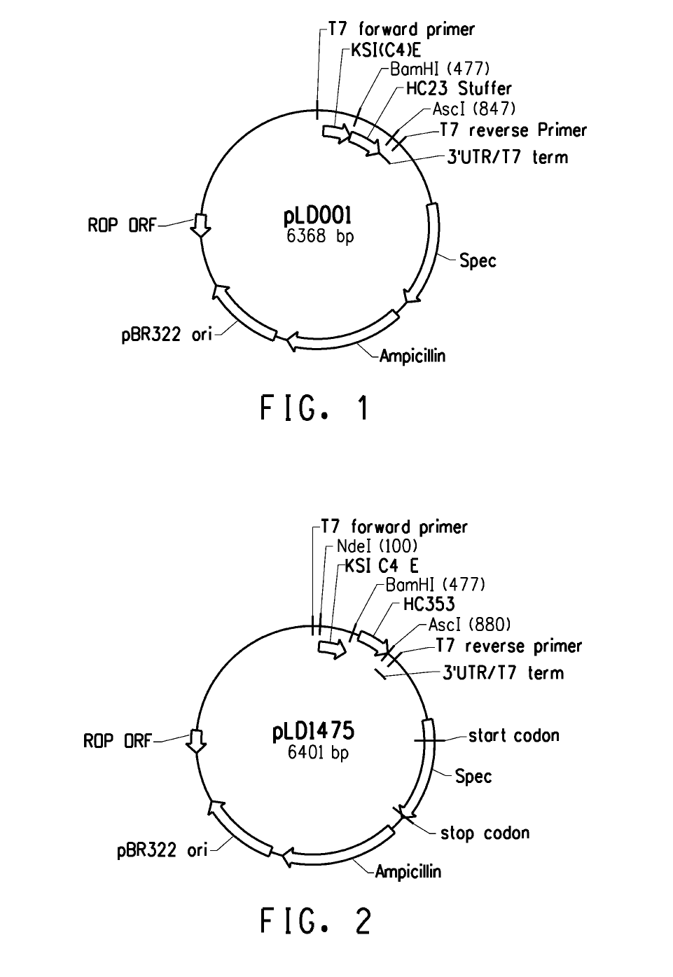

The cloned peptide-coding DNA sequence was recloned into the expression vector pLD001 (FIG. 1; SEQ ID NO: 48) for expression in E. coli. For that purpose, the coding sequence on a restriction endonuclease fragment bounded by BamHI and AscI sites was ligated between BamHI and AscI sites in pLD001 using standard recombinant DNA methods. The resulting gene fusion resulted in a gene product in which the HC353 coding sequence was fused downstream from a modified fragment of ketosteroid isomerase [(KSI(C4)E); SEQ ID NO: 49] that served to drive the peptide into insoluble inclusion bodies in E. coli (See U.S. Patent Application Publication Nos. US 2009-0029420 and US 2009-0043075).

The vector pLD001 was derived from the commercially available vector pDEST17 (Invitrogen, Carlsbad, Calif.). It includes sequences derived from the commercially available vector pET31b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31b was modified using standard mutagenesis procedures (QuickChange II, Stratagene, La Jolla, Calif.) to include three additional Cys codons, in addition to the one Cys codon found in the wild-type KSI sequence. In addition, all Asp codons in the coding sequence were replaced by Glu codons. The plasmid pLD001, given by SEQ ID NO: 48 was constructed using standard recombinant DNA methods, which are well known to those skilled in the art.

The DNA sequence (SEQ ID NO: 47) encoding peptide HC353 was inserted into pLD001 by substituting for sequences in the vector between the BamHI and AscI sites. Plasmid DNA containing the peptide encoding sequences and vector DNA were digested with endonuclease restriction enzymes BamHI and Ascl, then the peptide-encoding sequences and vector DNA were mixed and ligated by phage T4 DNA ligase using standard DNA cloning procedures, which are well known to those skilled in the art. Correct constructs, in which the sequences encoding the peptide HC353 were inserted into pLD001, were identified by restriction analysis and verified by DNA sequencing, using standard methods. The DNA sequence of the expression plasmid pLD1475 encoding the KSI(C4)E-HC353 peptide fusion is provided as SEQ ID NO: 50 (FIG. 2).

### Example 5

### Preparation, Isolation and Processing of Fusion Protein

### Growth Conditions

The BL21-Al E. coli cells containing the expression plasmid were grown for 20 hours at 37° C. with agitation (200 rpm) in 2.8-L Fernbach flasks containing 1-L of modified ZYP-5052 auto-induction media (Studier, F. William, Protein Expression and Purification (2005) 41:207-234). The media composition per liter was as follows: 10 g/L Tryptone, 5 g/L Yeast Extract, 5 g/L NaCl, 50 mM Na₂HPO₄, 50 mM KH₂PO₄, 25 mM (NH4)₂SO₄, 3 mM MgSO₄, 0.75% glycerol, 0.075% glucose and 0.05% Arabinose (inducer for BL21 Al T7 system). Under these conditions about 20 g/L wet weight of cells are obtained per liter.

### Inclusion Body Isolation

The entire process was performed in one 500-mL bottle. Cells were separated from the growth media by centrifugation and washed with 200-mL (10 g cell paste/100-mL buffer) 20 mM Tris buffer and 10 mM EDTA at pH 8.0. The cell paste was resuspended in 200-mL of 20 mM Tris buffer and 10 mM EDTA at pH 8.0 with added lysozyme (5 mg/200 mL) and taken through at lease one freeze-thaw cycles to facilitate lysis. Lysis was completed by sonication and the inclusion body paste was recovered by centrifugation (9000 RCF 20 minutes 4° C.). Each additional wash step included resuspension of the inclusion body paste, followed by sonication and centrifugation (9000 RCF 20 minutes 4° C.). Wash steps included a high pH wash (50 mM Tris HCL pH 9.0) followed by additional washes with 20 mM Tris-HCl pH 8.0. Typically 5 g/L inclusion body paste was recovered.

### Acid Cleavage

The recovered inclusion body paste was resuspended in 100-mL of pure water and the pH of the mixture adjusted to 2.2 using HCl. The acidified suspension was heated to 70° C. for 14 hours with agitation to complete cleavage of the DP site separating the fusion peptide from the product peptide.

Oxidative Cross-Linking to Separate the IBT from the Peptide of Interest

The product was cooled ˜5° C. then the pH neutralized to 5.3 using NaOH and cooled for an additional 1 hour at ˜5° C. to facilitate precipitation of cysteine cross-linked KSI (C4)E tag (see U.S. Patent Application Publication No. US 2009-0043075). The mixture was then centrifuged at 10000 RCF for 30 minutes 4° C. The resulting pellet contained the inclusion body fusion partner KSI (C4)E. The supernatant containing the peptide of interest was then lyophilized.

### Example 6

### 10-Liter Fermentation

The recombinant E. coli strain described above was grown in a 10-L fermentation, which was run in batch mode initially, and then in fed-batch mode. The composition of the fermentation medium is given in Table 5. The pH of the fermentation medium was 6.7. The fermentation medium was sterilized by autoclaving, after which the following sterilized components were added: thiamine hydrochloride (4.5 mg/L), glucose (22.1 g/L), trace elements, see Table 6 (10 mL/L), ampilcillin (100 mg/L), and inoculum (seed) (125 mL). The pH was adjusted as needed using ammonium hydroxide (20 vol %) or phosphoric acid (20 vol %). The added components were sterilized either by autoclaving or filtration.

TABLE 5
Composition of Fermentation Medium

| Component | Concentration | |
|---|---|---|
| KH₂PO₄ | 9 | g/L |
| (NH₄)₂HPO₄ | 4 | g/L |
| MgSO₄•7H₂O | 1.2 | g/L |
| Citric Acid | 1.7 | g/L |
| Yeast extract | 5.0 | g/L |
| Mazu DF 204 Antifoam | 0.1 | mL/L |

TABLE 6
Trace Elements

| Component | Concentration (mg/L) |
|---|---|
| EDTA | 840 |
| CoCl₂•H₂O | 250 |
| MnCl₂•4H₂O | 1500 |
| CuCl₂•2H₂O | 150 |
| H₃BO₃ | 300 |
| Na₂MoO₄•2H₂O | 250 |
| Zn(CH₃COO)₂•H₂O | 1300 |
| Ferric citrate | 10000 |

The operating conditions for the fermentation are summarized in Table 7. The initial concentration of glucose was 22.1 g/L. When the initial residual glucose was depleted, a pre-scheduled, exponential glucose feed was initiated starting the fed-batch phase of the fermentation run. The glucose feed (see Tables 8 and 9) contained 500 g/L of glucose and was supplemented with 5 g/L of yeast extract. The components of the feed medium were sterilized either by autoclaving or filtration. The goal was to sustain a specific growth rate of 0.13 h⁻¹, assuming a yield coefficient (biomass to glucose) of 0.25 g/g, and to maintain the acetic acid levels in the fermentation vessel at very low values (i.e., less than 0.2 g/L). The glucose feed continued until the end of the run. Induction was initiated with a bolus of 2 g/L of L-arabinose at the selected time (i.e., 15 h of elapsed fermentation time). A bolus to deliver 5 g of yeast extract per liter of fermentation broth was added to the fermentation vessel at the following times: 1 h prior to induction, at induction time, and 1 h after induction time. The fermentation run was terminated after 19.97 h of elapsed fermentation time, and 4.97 h after the induction time.

TABLE 7
Fermentation Operating Conditions

| Condition | Initial | Minimum | Maximum |
|---|---|---|---|
| Stirring speed | 220 rpm | 220 rpm | 1200 rpm |
| Air Flow | 3 SLPM | 3 SLPM | 30 SLPM |
| Temperature | 37° C. | 37° C. | 37° C. |
| pH | 6.7 | 6.7 | 6.7 |
| Pressure | 0.500 atm | 0.500 atm | 0.500 atm |
| | (50.7 kPa) | (50.7 kPa) | (50.7 kPa) |
| Dissolved O₂* | 20% | 20% | 20% |

*Cascade stirrer, then air flow.

TABLE 8
Composition of Feed Medium

| Component | Concentration | |
|---|---|---|
| MgSO₄•7H₂O | 2.0 | g/L |
| Glucose | 500 | g/L |
| Ampicillin | 150 | mg/L |
| (NH₄)₂HPO₄ | 4 | g/L |
| KH₂PO₄ | 9 | g/L |
| Yeast extract | 5.0 | g/L |
| Trace Elements - Feed (Table 9) | 10 | mL/L |

TABLE 9
Trace Elements - Feed

| Component | Concentration (mg/L) |
|---|---|
| EDTA | 1300 |
| CoCl₂•H₂O | 400 |
| MnCl₂•4H₂O | 2350 |
| CuCl₂•2H₂O | 250 |
| H₃BO₃ | 500 |
| Na₂MoO₄•2H₂O | 400 |
| Zn(CH₃COO)₂•H₂O | 1600 |
| Ferric citrate | 4000 |

### Isolation and Purification of Peptides:

After completion of the fermentation run, the entire fermentation broth was passed three times through an APV model 1000 Gaulin type homogenizer at 12,000 psi (82,700 kPa). The broth was cooled to below 5° C. prior to each homogenization. The homogenized broth was immediately processed through a Westfalia WHISPERFUGE™ (Westfalia Separator Inc., Northvale, N.J.) stacked disc centrifuge at 600 mL/min and 12,000 RCF to separate inclusion bodies from suspended cell debris and dissolved impurities. The recovered paste was resuspended at 15 g/L (dry basis) in water and the pH was adjusted to a value between 8.0 and 10.0 using NaOH. The pH was chosen to help remove cell debris from the inclusion bodies without dissolving the inclusion body proteins. The suspension was passed through the APV 1000 Gaulin type homogenizer at 12,000 psi (82,700 kPa) for a single pass to provide rigorous mixing. The homogenized high pH suspension was immediately processed in a Westfalia WHISPERFUGE™ stacked disc centrifuge at 600 mL/min and 12,000 RCF to separate the washed inclusion bodies from suspended cell debris and dissolved impurities. The recovered paste was resuspended at 15 gm/L (dry basis) in pure water. The suspension was passed through the APV 1000 Gaulin type homogenizer at 12,000 psi (82,700 kPa) for a single pass to provide rigorous washing. The homogenized suspension was immediately processed in a Westfalia WHISPERFUGE™ stacked disc centrifuge at 600 mL/min and 12,000 RCF to separate the washed inclusion bodies from residual suspended cell debris and NaOH.

The recovered paste was resuspended in pure water at 25 g/L (dry basis) and the pH of the mixture was adjusted to 2.2 using HCl. The acidified suspension was heated to 70° C. for 5 to 14 h to complete cleavage of the DP site separating the fusion peptide from the product peptide without damaging the target peptide. The product slurry was adjusted to pH 5.24 using NaOH and then was cooled to 5° C. and held for 12 h. The mixture was centrifuged at 9000 RCF for 30 min and the supernatant was decanted. The supernatant was then filtered with a 0.2 μm membrane and lyophilized.

The peptide product was characterized by reversed-phase liquid chromatography and mass spectroscopy and show to have the expected molecular weight. The peptide HC353 comprised 41.3% (w/w) of the lyophilized material. Most of the remaining mass was salt.

### Example 7

### Performance of HC353 for Uptake and Retention of an Iron Oxide-Based Pigment

The purpose of this example is to illustrate a sequential treatment coloring method using HC353 and to illustrate the color retention of iron oxide-based pigment on hair after a shampoo cycle. The hair was pre-treated with peptide HC353 and subsequently with the iron oxide-based pigment.

### Preparation of Small Hair Tress

A 2-3 mm wide strip of a polyurethane-based adhesive (e.g. 3M SCOTCH-GRIP™ 4475 Plastic Adhesive) was placed on a TEFLON® sheet (E.I. duPont de Nemours and Company, Inc., Wilmington, Del.). Hair to be tufted was spread out to 2-3 mm thickness and placed over the glue. Another 1-2 mm wide strip of adhesive was placed on the top side and glue-line was pressed down using a TEFLON®-covered metal bar to a thickness of 1-1.5 mm. The adhesive was dried for 6-12 hours. Hair sample were peeled off and cut approximately 1.5 to 2.0 cm away from the glue-line. The swatches were cut to 5-6 mm width to yield tufts of 60-80 mg hair.
Step-1: Pretreatment with peptide. HC353 (0.0025 micromoles) was dissolved in 0.5 mL buffer (25 mM tris.HCl, 250 mM NaCl, pH 7.5). A small tress of natural white hair (International Hair Importers) was suspended in the peptide solution in a vial and agitated at a low speed on a vortex mixer for 30 minutes. The tress was rinsed with the treatment-buffer twice followed by a thorough rinse under a jet of de-ionized water.
Step-2: Pigment application. The peptide-pretreated tress was treated with a 0.25% iron oxide pigment dispersion in 25 mM tris.HCl in a vial at slow agitation. After 30 minutes the tress was thoroughly rinsed under a jet of deionized water and dried in air. The L*, a* and b* values for color uptake was measured using a spectrophotometer.
Step-3: Shampoo cycle. The tresses subjected to shampoo cycle were placed in wells of a 24-well plate. Glass and stainless steel beads (3 mm glass beads (4), 4 mm stain steel beads (1), 6.35 mm glass beads (2) were charged into each well. Approximately 1.0-mL of 0.2% sodium lauryl ether sulfate (SLES) solution was added to each well. The well plate was covered with a flexible SANTOPRENE® mat and was agitated at high speed on the vortex mixer for 30 sec. The shampoo was removed from the wells by suction. Approximately 4-mL of de-ionized water was added to each well; the plate was agitated at a low speed on the vortex mixer for 5-10 sec. The rinse solution was removed by suction. The tress was thoroughly rinsed under a jet of de-ionized water and subjected to the next shampoo cycle. After the last shampoo cycle, the tress was dried in air and the retained color is measured.
Delta-E values are calculated from L*, a* and b* using the formula

$$Δ E uptake = \sqrt{( ( {Lu}^{*} - L 0 ) \hat{} 2 + ( a {u}^{*} - a 0 ) \hat{} 2 + ( {bu}^{*} - b ) \hat{} 2 )}$$

$$and$$

$$Δ E retention = \sqrt{( ( {Lr}^{*} - L 0 ) \hat{} 2 + ( {ar}^{*} - a 0 ) \hat{} 2 + ( {br}^{*} - b 0 ) \hat{} 2 )}$$

where,
Lu*, au* and bu* are L*, a* and b* values for a sample tress after color uptake,
Lr*, ar* and br* are L*, a* and b* values for a sample tress after shampoo cycles, and
L0*, a0* and b0* are L*, a* and b* values for untreated natural white hair.

The L* (Lu* or Lr*)=the lightness variable and a* (au* or ar*) and b* (bu* or br*) are the chromaticity coordinates of CIELAB colorspace as defined by the International Commission of Illumination (CIE) (Minolta, Precise Color Communication—Color Control From Feeling to Instrumentation, Minolta Camera Co., 1996). Larger Delta E value are indicative of better color retention. The results are provided in Table 10.

TABLE 10
Performance of HC353 Using Sequential Application Method

| | | | Buffer, | | |
|---|---|---|---|---|---|
| | Peptide | Peptide | tris•HCl | | |
| | (SEQ ID | amount, | mM/salt, | ΔE | ΔE |
| Experiment | NO: 46) | μmoles | mM | uptake | retention |
| 1 | HC353 | 0.0125 | 25/12 | 33 | 26 |
| 2 | HC353 | 0.0125 | 25/61 | 33 | 25 |
| 3 | HC353 | 0.0125 | 25/166 | 31 | 26 |
| 4 | HC353 | 0.005 | 25/250 | 23 | — |
| 5 | HC353 | 0.0025 | 25/250 | 25 | — |
| 6 | HC354 | 0.0125 | 25/10 | 32 | 24 |
| 7 | HC354 | 0.0125 | 25/61 | 33 | 27 |
| 8 | No | — | 25/250 | 2 | — |
| | peptide | | | | |

What is claimed is:

1. An iron oxide-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38.

2. A peptide-based reagent selected from the group consisting of:
a) a single chain peptide-based reagent having the general structure:
[(BSBP)ₘ-(IOBP)ₙ]ₓ; and
b) a single chain peptide-based reagent having the general structure:
[[(BSBP)ₘ-S_{q}]ₓ-[(IOBP)ₙ-Sᵣ]_{z}]_{y},;
wherein
i) BSBP is a body surface-binding peptide;
ii) IOBP is an iron oxide-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, and 38;
iii) S is a spacer;
iv) m, n, x and z independently range from 1 to about 10;
v) y is from 1 to 5; and
vi) q an r are each independently 0 or 1, provided that both r and q may not be 0.

3. The peptide-based reagent according to claim 2 wherein the body surface-binding peptide is from about 7 to about 60 amino acids.

4. The peptide-based reagent according to claim 2 wherein the spacer is a peptide linker or a peptide bridge comprising a length of 1 amino acid to 60 amino acids.

5. The peptide-based reagent according to claim 3 wherein the body surface-binding peptide binds to a body surface selected from the group consisting of hair, skin, nail, and tooth.

6. The peptide-based reagent according to claim 2 wherein the iron oxide-binding peptide has affinity for an iron oxide-based pigment comprising ferric oxide, ferrous ferric oxide, or mixtures thereof.

7. The peptide-based reagent of claim 2 wherein the spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, ethyl alkyl chains, propyl alkyl chains, hexyl alkyl chains, steryl alkyl chains, cetyl alkyl chains, and palmitoyl alkyl chains.

8. The peptide-based reagent of claim 2 wherein the spacer is a peptide linker comprising a length of 1 amino acid to 60 amino acids.

9. The peptide-based reagent according to claim 2 wherein the peptide-based reagent is from about 14 to about 600 amino acids in length.

10. A personal care composition comprising the iron oxide-binding peptide of claim 1 or the peptide-based reagent of claim 2 and at least one iron oxide-based pigment.

11. A method for coloring a body surface comprising:
a) providing at least one iron oxide-based pigment;
b) providing a composition comprising the peptide-based reagent according to claim 2; and
c) applying said at least one iron oxide pigment of (a) with the composition of (b) to a body surface for a time sufficient for the peptide-based reagent to bind to the iron oxide-based pigment and the body surface.

12. The method according to claim 11 wherein the body surface is selected from the group consisting of hair, skin, nail, and tooth.

13. The method according to claim 11 further comprising the step of:
d) applying a composition comprising a polymeric sealant to the body surface subsequent to step (c).

14. The method according to claim 13 wherein the polymeric sealant is selected from the group consisting of poly(allylamine), acrylates, acrylate copolymers, polyurethanes, carbomers, methicones, amodimethicones, polyethylenene glycol, beeswax, and siloxanes.


SEQUENCE LISTING (269 sequences)

SEQ ID NO: 1 - protein, 12 aa, artificial sequence
  synthetic construct
WAPEKDHMQLMK

SEQ ID NO: 2 - protein, 12 aa, artificial sequence
  synthetic construct
WAPEKDYMQLMK

SEQ ID NO: 3 - protein, 20 aa, artificial sequence
  synthetic construct
CPLDTPTHKTKHEYKTRCRH

SEQ ID NO: 4 - protein, 18 aa, artificial sequence
  synthetic construct
DHDHPRLHKRQEKSEHLH

SEQ ID NO: 5 - protein, 20 aa, artificial sequence
  synthetic construct
DSHHNHHKQDSRPQHRKTPN

SEQ ID NO: 6 - protein, 15 aa, artificial sequence
  synthetic construct
EGGNAPHHKPHHRKH

SEQ ID NO: 7 - protein, 18 aa, artificial sequence
  synthetic construct
HDSHRPLTQHGHRHSHVP

SEQ ID NO: 8 - protein, 18 aa, artificial sequence
  synthetic construct
HDSNHCSHSTRRPNCART

SEQ ID NO: 9 - protein, 15 aa, artificial sequence
  synthetic construct
ATRVDNTPASNPPSL

SEQ ID NO: 10 - protein, 15 aa, artificial sequence
  synthetic construct
DGIKPFHLMTPTLAN

SEQ ID NO: 11 - protein, 18 aa, artificial sequence
  synthetic construct
DITPPGSTHHRKPHRHQH

SEQ ID NO: 12 - protein, 15 aa, artificial sequence
  synthetic construct
DNLWPQPLNVEDDRY

SEQ ID NO: 13 - protein, 18 aa, artificial sequence
  synthetic construct
ENEKHRHNTHEALHSHFK

SEQ ID NO: 14 - protein, 20 aa, artificial sequence
  synthetic construct
GAIWPASSALMTEHNPTDNH

SEQ ID NO: 15 - protein, 15 aa, artificial sequence
  synthetic construct
GDTNQDTVMWYYTVN

SEQ ID NO: 16 - protein, 15 aa, artificial sequence
  synthetic construct
HNGPYGMLSTGKIHF

SEQ ID NO: 17 - protein, 15 aa, artificial sequence
  synthetic construct
LDGGYRDTPDNYLKG

SEQ ID NO: 18 - protein, 15 aa, artificial sequence
  synthetic construct
LHTKTENSHTNMKTT

SEQ ID NO: 19 - protein, 20 aa, artificial sequence
  synthetic construct
NAQYDPPTLNKGAVRKAAST

SEQ ID NO: 20 - protein, 15 aa, artificial sequence
  synthetic construct
NGNNHTDIPNRSSYT

SEQ ID NO: 21 - protein, 18 aa, artificial sequence
  synthetic construct
QSTNHHHPHAKHPRVNTH

SEQ ID NO: 22 - protein, 15 aa, artificial sequence
  synthetic construct
SNNDYVGTYPATAIQ

SEQ ID NO: 23 - protein, 15 aa, artificial sequence
  synthetic construct
STQHNLHDRNIYFVS

SEQ ID NO: 24 - protein, 20 aa, artificial sequence
  synthetic construct
TANNKTPAGAPNAAVGLAQR

SEQ ID NO: 25 - protein, 15 aa, artificial sequence
  synthetic construct
TEPTRISNYRSIPND

SEQ ID NO: 26 - protein, 18 aa, artificial sequence
  synthetic construct
THNPREHARHHHHNEYKH

SEQ ID NO: 27 - protein, 15 aa, artificial sequence
  synthetic construct
THPPCWYETNCIVQE

SEQ ID NO: 28 - protein, 20 aa, artificial sequence
  synthetic construct
TTNPHKPASHHHDHRPALRH

SEQ ID NO: 29 - protein, 15 aa, artificial sequence
  synthetic construct
WLVADNATDGHSHQK

SEQ ID NO: 30 - protein, 15 aa, artificial sequence
  synthetic construct
YTDSMSDQTPEFAKY

SEQ ID NO: 31 - protein, 13 aa, artificial sequence
  synthetic construct
WAPEKDHMQLMKK

SEQ ID NO: 32 - protein, 13 aa, artificial sequence
  synthetic construct
WAPEKDYMQLMKK

SEQ ID NO: 33 - protein, 21 aa, artificial sequence
  synthetic construct
CPLDTPTHKTKHEYKTRCRHK

SEQ ID NO: 34 - protein, 19 aa, artificial sequence
  synthetic construct
DHDHPRLHKRQEKSEHLHK

SEQ ID NO: 35 - protein, 21 aa, artificial sequence
  synthetic construct
DSHHNHHKQDSRPQHRKTPNK

SEQ ID NO: 36 - protein, 16 aa, artificial sequence
  synthetic construct
EGGNAPHHKPHHRKHK

SEQ ID NO: 37 - protein, 19 aa, artificial sequence
  synthetic construct
HDSHRPLTQHGHRHSHVPK

SEQ ID NO: 38 - protein, 19 aa, artificial sequence
  synthetic construct
HDSNHCSHSTRRPNCARTK

SEQ ID NO: 39 - DNA, 20 bp, artificial sequence
  primer
ccctcatagttagcgtaacg

SEQ ID NO: 40 - protein, 20 aa, artificial sequence
  synthetic hair-binding peptide
AQSQLPDKHSGLHERAPQRY

SEQ ID NO: 41 - protein, 15 aa, artificial sequence
  synthetic hair-binding peptide
HDHKNQKETHQRHAA

SEQ ID NO: 42 - protein, 33 aa, artificial sequence
  synthetic linker
EPEPEPEPIPEPPKEAPVVIEKPKPKPKPKPKP

SEQ ID NO: 43 - protein, 76 aa, artificial sequence
  synthetic construct
PSAQSQLPDKHSGLHERAPQRYGPEPEPEPEPIPEPPKEAPVVIEKPKPKPKPKPKPPAHDHKNQKETHQRHAAGS

SEQ ID NO: 44 - protein, 8 aa, artificial sequence
  synthetic peptide bridge
GSGGGGSP

SEQ ID NO: 45 - protein, 21 aa, artificial sequence
  synthetic peptide linker
GKGKGKGKGKGKGKGKGKGKG

SEQ ID NO: 46 - protein, 129 aa, artificial sequence
  synthetic peptide-based reagent HC353
PSAQSQLPDKHSGLHERAPQRYGPEPEPEPEPIPEPPKEAPVVIEKPKPKPKPKPKPPAHDHKNQKETHQRHAAGSGGGGSPWAPEKDHMQLMKGKGKGKGKGKGKGKGKGKGKGWAPEKDHMQLMKGK

SEQ ID NO: 47 - DNA, 393 bp, artificial sequence
  synthetic nucleic acid sequence encoding HC353
ccttctgctcaatctcaactgcctgataaacattctggtctgcacgagcgcgctccgcagcgctatggccctgaaccggaacctgagccagagccgattccggaaccgccgaaagaggcgccagtagttatcgaaaaacctaaaccaaaaccaaaaccgaaaccgaaacctccggcccacgaccacaaaaaccagaaagaaacccatcagcgtcacgccgctggttctggtggtggcggtagcccgtgggctccggaaaaggatcacatgcagctgatgaaaggcaaaggtaagggcaaaggtaaaggtaagggtaaaggcaaaggcaaaggcaagggcaagggttgggcaccagagaaagaccacatgcaactgatgaagggtaaataatga

SEQ ID NO: 48 - DNA, 6368 bp, artificial sequence
  synthetic construct plasmid
agatctcgatcccgcgaaattaatacgactcactatagggagaccacaacggtttccctctagaaataattttgtttaactttaagaaggagatatacatatgcacactccagaacatatcaccgcagtagtacagcgttttgtggcagctctgaacgcgggcgagctggaaggtattgtggcgctgttcgcggaagaagccaccgtggaagaaccggtgggttctgaaccgcgttccggcaccgcagcctgccgtgaattttacgcaaacagcctgaagctgccgctggcggttgaactgacccaagaatgtcgtgcggtggctaacgaagccgctttcgcgttcaccgtgtccttcgaataccagggtcgtaagaccgttgtggcgccatgcgaacactttcgtttcaacggcgcaggcaaagtggtttccatccgcgcactgttcggtgaaaagaacatccatgcttgtcagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 49 - protein, 127 aa, artificial sequence
  synthetic construct inclusion body tag KSI(C4)E
MHTPEHITAVVQRFVAALNAGELEGIVALFAEEATVEEPVGSEPRSGTAACREFYANSLKLPLAVELTQECRAVANEAAFAFTVSFEYQGRKTVVAPCEHFRFNGAGKVVSIRALFGEKNIHACQGS

SEQ ID NO: 50 - DNA, 6401 bp, artificial sequence
  synthetic construct plasmid
agatctcgatcccgcgaaattaatacgactcactatagggagaccacaacggtttccctctagaaataattttgtttaactttaagaaggagatatacatatgcacactccagaacatatcaccgcagtagtacagcgttttgtggcagctctgaacgcgggcgagctggaaggtattgtggcgctgttcgcggaagaagccaccgtggaagaaccggtgggttctgaaccgcgttccggcaccgcagcctgccgtgaattttacgcaaacagcctgaagctgccgctggcggttgaactgacccaagaatgtcgtgcggtggctaacgaagccgctttcgcgttcaccgtgtccttcgaataccagggtcgtaagaccgttgtggcgccatgcgaacactttcgtttcaacggcgcaggcaaagtggtttccatccgcgcactgttcggtgaaaagaacatccatgcttgtcagggatccgatccttctgctcaatctcaactgcctgataaacattctggtctgcacgagcgcgctccgcagcgctatggccctgaaccggaacctgagccagagccgattccggaaccgccgaaagaggcgccagtagttatcgaaaaacctaaaccaaaaccaaaaccgaaaccgaaacctccggcccacgaccacaaaaaccagaaagaaacccatcagcgtcacgccgctggttctggtggtggcggtagcccgtgggctccggaaaaggatcacatgcagctgatgaaaggcaaaggtaagggcaaaggtaaaggtaagggtaaaggcaaaggcaaaggcaagggcaagggttgggcaccagagaaagaccacatgcaactgatgaagggtaaataatgaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 51 - protein, 12 aa, artificial sequence
  synthetic hair-binding peptide
RVPNKTVTVDGA

SEQ ID NO: 52 - protein, 12 aa, artificial sequence
  Synthetic construct
DRHKSKYSSTKS

SEQ ID NO: 53 - protein, 12 aa, artificial sequence
  Synthetic construct
KNFPQQKEFPLS

SEQ ID NO: 54 - protein, 12 aa, artificial sequence
  Synthetic construct
QRNSPPAMSRRD

SEQ ID NO: 55 - protein, 12 aa, artificial sequence
  Synthetic construct
TRKPNMPHGQYL

SEQ ID NO: 56 - protein, 12 aa, artificial sequence
  Synthetic construct
KPPHLAKLPFTT

SEQ ID NO: 57 - protein, 12 aa, artificial sequence
  Synthetic construct
NKRPPTSHRIHA

SEQ ID NO: 58 - protein, 12 aa, artificial sequence
  Synthetic construct
NLPRYQPPCKPL

SEQ ID NO: 59 - protein, 12 aa, artificial sequence
  Synthetic construct
RPPWKKPIPPSE

SEQ ID NO: 60 - protein, 12 aa, artificial sequence
  Synthetic construct
RQRPKDHFFSRP

SEQ ID NO: 61 - protein, 12 aa, artificial sequence
  Synthetic construct
SVPNKXVTVDGX

SEQ ID NO: 62 - protein, 12 aa, artificial sequence
  Synthetic construct
TTKWRHRAPVSP

SEQ ID NO: 63 - protein, 12 aa, artificial sequence
  Synthetic construct
WLGKNRIKPRAS

SEQ ID NO: 64 - protein, 12 aa, artificial sequence
  Synthetic construct
SNFKTPLPLTQS

SEQ ID NO: 65 - protein, 12 aa, artificial sequence
  Synthetic construct
SVSVGMKPSPRP

SEQ ID NO: 66 - protein, 7 aa, artificial sequence
  Synthetic construct
DLHTVYH

SEQ ID NO: 67 - protein, 7 aa, artificial sequence
  Synthetic construct
HIKPPTR

SEQ ID NO: 68 - protein, 7 aa, artificial sequence
  Synthetic construct
HPVWPAI

SEQ ID NO: 69 - protein, 7 aa, artificial sequence
  Synthetic construct
MPLYYLQ

SEQ ID NO: 70 - protein, 26 aa, artificial sequence
  Synthetic construct
HLTVPWRGGGSAVPFYSHSQITLPNH

SEQ ID NO: 71 - protein, 41 aa, artificial sequence
  Synthetic construct
GPHDTSSGGVRPNLHHTSKKEKRENRKVPFYSHSVTSRGNV

SEQ ID NO: 72 - protein, 7 aa, artificial sequence
  Synthetic construct
KHPTYRQ

SEQ ID NO: 73 - protein, 7 aa, artificial sequence
  Synthetic construct
HPMSAPR

SEQ ID NO: 74 - protein, 7 aa, artificial sequence
  Synthetic construct
MPKYYLQ

SEQ ID NO: 75 - protein, 7 aa, artificial sequence
  Synthetic construct
MHAHSIA

SEQ ID NO: 76 - protein, 12 aa, artificial sequence
  Synthetic construct
AKPISQHLQRGS

SEQ ID NO: 77 - protein, 12 aa, artificial sequence
  Synthetic construct
APPTPAAASATT

SEQ ID NO: 78 - protein, 12 aa, artificial sequence
  Synthetic construct
DPTEGARRTIMT

SEQ ID NO: 79 - protein, 12 aa, artificial sequence
  Synthetic construct
LDTSFPPVPFHA

SEQ ID NO: 80 - protein, 12 aa, artificial sequence
  Synthetic construct
LDTSFHQVPFHQ

SEQ ID NO: 81 - protein, 11 aa, artificial sequence
  Synthetic construct
LPRIANTWSPS

SEQ ID NO: 82 - protein, 12 aa, artificial sequence
  Synthetic construct
RTNAADHPAAVT

SEQ ID NO: 83 - protein, 12 aa, artificial sequence
  Synthetic construct
SLNWVTIPGPKI

SEQ ID NO: 84 - protein, 12 aa, artificial sequence
  Synthetic construct
TDMQAPTKSYSN

SEQ ID NO: 85 - protein, 12 aa, artificial sequence
  Synthetic construct
TIMTKSPSLSCG

SEQ ID NO: 86 - protein, 12 aa, artificial sequence
  Synthetic construct
TPALDGLRQPLR

SEQ ID NO: 87 - protein, 12 aa, artificial sequence
  Synthetic construct
TYPASRLPLLAP

SEQ ID NO: 88 - protein, 12 aa, artificial sequence
  Synthetic construct
AKTHKHPAPSYS

SEQ ID NO: 89 - protein, 12 aa, artificial sequence
  Synthetic construct
TDPTPFSISPER

SEQ ID NO: 90 - protein, 12 aa, artificial sequence
  Synthetic construct
SQNWQDSTSYSN

SEQ ID NO: 91 - protein, 12 aa, artificial sequence
  Synthetic construct
WHDKPQNSSKST

SEQ ID NO: 92 - protein, 12 aa, artificial sequence
  Synthetic construct
LDVESYKGTSMP

SEQ ID NO: 93 - protein, 7 aa, artificial sequence
  Synthetic construct
NTPKENW

SEQ ID NO: 94 - protein, 7 aa, artificial sequence
  Synthetic construct
NTPASNR

SEQ ID NO: 95 - protein, 7 aa, artificial sequence
  Synthetic construct
PRGMLST

SEQ ID NO: 96 - protein, 7 aa, artificial sequence
  Synthetic construct
PPTYLST

SEQ ID NO: 97 - protein, 12 aa, artificial sequence
  Synthetic construct
TIPTHRQHDYRS

SEQ ID NO: 98 - protein, 7 aa, artificial sequence
  Synthetic construct
TPPTHRL

SEQ ID NO: 99 - protein, 7 aa, artificial sequence
  Synthetic construct
LPTMSTP

SEQ ID NO: 100 - protein, 7 aa, artificial sequence
  Synthetic construct
LGTNSTP

SEQ ID NO: 101 - protein, 12 aa, artificial sequence
  Synthetic construct
TPLTGSTNLLSS

SEQ ID NO: 102 - protein, 7 aa, artificial sequence
  Synthetic construct
TPLTKET

SEQ ID NO: 103 - protein, 7 aa, artificial sequence
  Synthetic construct
KQSHNPP

SEQ ID NO: 104 - protein, 7 aa, artificial sequence
  Synthetic construct
QQSHNPP

SEQ ID NO: 105 - protein, 7 aa, artificial sequence
  Synthetic construct
TQPHNPP

SEQ ID NO: 106 - protein, 12 aa, artificial sequence
  Synthetic construct
STNLLRTSTVHP

SEQ ID NO: 107 - protein, 12 aa, artificial sequence
  Synthetic construct
HTQPSYSSTNLF

SEQ ID NO: 108 - protein, 7 aa, artificial sequence
  Synthetic construct
SLLSSHA

SEQ ID NO: 109 - protein, 12 aa, artificial sequence
  Synthetic construct
QQSSISLSSHAV

SEQ ID NO: 110 - protein, 7 aa, artificial sequence
  Synthetic construct
NASPSSL

SEQ ID NO: 111 - protein, 7 aa, artificial sequence
  Synthetic construct
HSPSSLR

SEQ ID NO: 112 - protein, 7 aa, artificial sequence
  Synthetic construct
KXSHHTH

SEQ ID NO: 113 - protein, 7 aa, artificial sequence
  Synthetic construct
EXSHHTH

SEQ ID NO: 114 - protein, 12 aa, artificial sequence
  Synthetic construct
SHHTHYGQPGPV

SEQ ID NO: 115 - protein, 7 aa, artificial sequence
  Synthetic construct
LESTSLL

SEQ ID NO: 116 - protein, 7 aa, artificial sequence
  Synthetic construct
DLTLPFH

SEQ ID NO: 117 - protein, 8 aa, artificial sequence
  Synthetic construct
RTNAADHP

SEQ ID NO: 118 - protein, 12 aa, artificial sequence
  Synthetic construct
IPWWNIRAPLNA

SEQ ID NO: 119 - protein, 18 aa, artificial sequence
  Synthetic construct
EQISGSLVAAPWEGEGER

SEQ ID NO: 120 - protein, 12 aa, artificial sequence
  synthetic hair-binding peptide
TPPELLHGAPRS

SEQ ID NO: 121 - protein, 18 aa, artificial sequence
  Synthetic construct
LDTSFHQVPFHQKRKRKD

SEQ ID NO: 122 - protein, 18 aa, artificial sequence
  Synthetic construct
EQISGSLVAAPWKRKRKD

SEQ ID NO: 123 - protein, 18 aa, artificial sequence
  Synthetic construct
TPPELLHGDPRSKRKRKD

SEQ ID NO: 124 - protein, 13 aa, artificial sequence
  Synthetic construct
NTSQLSTEGEGED

SEQ ID NO: 125 - protein, 13 aa, artificial sequence
  Synthetic construct
TPPELLHGDPRSC

SEQ ID NO: 126 - protein, 20 aa, artificial sequence
  synthetic hair-binding peptide
HINKTNPHQGNHHSEKTQRQ

SEQ ID NO: 127 - protein, 15 aa, artificial sequence
  Synthetic construct
HAHKNQKETHQRHAA

SEQ ID NO: 128 - protein, 15 aa, artificial sequence
  Synthetic construct
HEHKNQKETHQRHAA

SEQ ID NO: 129 - protein, 20 aa, artificial sequence
  Synthetic construct
HNHMQERYTEPQHSPSVNGL

SEQ ID NO: 130 - protein, 17 aa, artificial sequence
  Synthetic construct
THSTHNHGSPRHTNADA

SEQ ID NO: 131 - protein, 20 aa, artificial sequence
  synthetic hair-binding peptide
GSCVDTHKADSCVANNGPAT

SEQ ID NO: 132 - protein, 20 aa, artificial sequence
  Synthetic construct
AQSQLPAKHSGLHERAPQRY

SEQ ID NO: 133 - protein, 20 aa, artificial sequence
  Synthetic construct
AQSQLPEKHSGLHERAPQRY

SEQ ID NO: 134 - protein, 20 aa, artificial sequence
  synthetic hair-binding peptide
TDMMHNHSDNSPPHRRSPRN

SEQ ID NO: 135 - protein, 20 aa, artificial sequence
  synthetic hair-binding peptide
TPPELAHTPHHLAQTRLTDR

SEQ ID NO: 136 - protein, 12 aa, artificial sequence
  Synthetic construct
RLLRLLRLLRLL

SEQ ID NO: 137 - protein, 12 aa, artificial sequence
  Synthetic construct
TPPELLHGEPRS

SEQ ID NO: 138 - protein, 12 aa, artificial sequence
  Synthetic construct
TPPELLHGAPRS

SEQ ID NO: 139 - protein, 12 aa, artificial sequence
  Synthetic construct
EQISGSLVAAPW

SEQ ID NO: 140 - protein, 12 aa, artificial sequence
  Synthetic construct
NEVPARNAPWLV

SEQ ID NO: 141 - protein, 13 aa, artificial sequence
  Synthetic construct
NSPGYQADSVAIG

SEQ ID NO: 142 - protein, 12 aa, artificial sequence
  Synthetic construct
AKPISQHLQRGS

SEQ ID NO: 143 - protein, 12 aa, artificial sequence
  Synthetic construct
LDTSFPPVPFHA

SEQ ID NO: 144 - protein, 12 aa, artificial sequence
  Synthetic construct
SLNWVTIPGPKI

SEQ ID NO: 145 - protein, 12 aa, artificial sequence
  Synthetic construct
TQDSAQKSPSPL

SEQ ID NO: 146 - protein, 12 aa, artificial sequence
  Synthetic construct
KELQTRNVVQRE

SEQ ID NO: 147 - protein, 12 aa, artificial sequence
  Synthetic construct
QRNSPPAMSRRD

SEQ ID NO: 148 - protein, 12 aa, artificial sequence
  Synthetic construct
TPTANQFTQSVP

SEQ ID NO: 149 - protein, 12 aa, artificial sequence
  Synthetic construct
AAGLSQKHERNR

SEQ ID NO: 150 - protein, 12 aa, artificial sequence
  Synthetic construct
ETVHQTPLSDRP

SEQ ID NO: 151 - protein, 12 aa, artificial sequence
  Synthetic construct
KNFPQQKEFPLS

SEQ ID NO: 152 - protein, 12 aa, artificial sequence
  Synthetic construct
LPALHIQRHPRM

SEQ ID NO: 153 - protein, 12 aa, artificial sequence
  Synthetic construct
QPSHSQSHNLRS

SEQ ID NO: 154 - protein, 12 aa, artificial sequence
  Synthetic construct
RGSQKSKPPRPP

SEQ ID NO: 155 - protein, 12 aa, artificial sequence
  Synthetic construct
THTQKTPLLYYH

SEQ ID NO: 156 - protein, 12 aa, artificial sequence
  Synthetic construct
TKGSSQAILKST

SEQ ID NO: 157 - protein, 7 aa, artificial sequence
  Synthetic construct
TAATTSP

SEQ ID NO: 158 - protein, 7 aa, artificial sequence
  Synthetic construct
LGIPQNL

SEQ ID NO: 159 - protein, 20 aa, artificial sequence
  Synthetic construct
THSTHNHGSPRHTNADAGNP

SEQ ID NO: 160 - protein, 20 aa, artificial sequence
  Synthetic construct
QQHKVHHQNPDRSTQDAHHS

SEQ ID NO: 161 - protein, 15 aa, artificial sequence
  Synthetic construct
HHGTHHNATKQKNHV

SEQ ID NO: 162 - protein, 15 aa, artificial sequence
  Synthetic construct
STLHKYKSQDPTPHH

SEQ ID NO: 163 - protein, 12 aa, artificial sequence
  Synthetic construct
SVSVGMKPSPRP

SEQ ID NO: 164 - protein, 12 aa, artificial sequence
  synthetic hair-binding peptide
TPPTNVLMLATK

SEQ ID NO: 165 - protein, 12 aa, artificial sequence
  Synthetic construct
TPPELLHGDPRS

SEQ ID NO: 166 - protein, 7 aa, artificial sequence
  synthetic hair-binding peptide
NTSQLST

SEQ ID NO: 167 - protein, 15 aa, artificial sequence
  Synthetic construct
STLHKYKSQDPTPHH

SEQ ID NO: 168 - protein, 12 aa, artificial sequence
  synthetic hair-binding peptide
GMPAMHWIHPFA

SEQ ID NO: 169 - protein, 20 aa, artificial sequence
  Synthetic construct
HNHMQERYTDPQHSPSVNGL

SEQ ID NO: 170 - protein, 20 aa, artificial sequence
  synthetic hair-binding peptide
TAEIQSSKNPNPHPQRSWTN

SEQ ID NO: 171 - protein, 12 aa, artificial sequence
  Synthetic construct
KRGRHKRPKRHK

SEQ ID NO: 172 - protein, 7 aa, artificial sequence
  Synthetic construct
RLLRLLR

SEQ ID NO: 173 - protein, 12 aa, artificial sequence
  Synthetic construct
HKPRGGRKKALH

SEQ ID NO: 174 - protein, 18 aa, artificial sequence
  Synthetic construct
KPRPPHGKKHRPKHRPKK

SEQ ID NO: 175 - protein, 18 aa, artificial sequence
  Synthetic construct
RGRPKKGHGKRPGHRARK

SEQ ID NO: 176 - protein, 12 aa, artificial sequence
  Synthetic construct
TPFHSPENAPGS

SEQ ID NO: 177 - protein, 13 aa, artificial sequence
  Synthetic construct
TPFHSPENAPGSK

SEQ ID NO: 178 - protein, 16 aa, artificial sequence
  Synthetic construct
TPFHSPENAPGSGGGS

SEQ ID NO: 179 - protein, 17 aa, artificial sequence
  Synthetic construct
TPFHSPENAPGSGGGSS

SEQ ID NO: 180 - protein, 15 aa, artificial sequence
  Synthetic construct
TPFHSPENAPGSGGG

SEQ ID NO: 181 - protein, 7 aa, artificial sequence
  Synthetic construct
FTQSLPR

SEQ ID NO: 182 - protein, 12 aa, artificial sequence
  Synthetic construct
KQATFPPNPTAY

SEQ ID NO: 183 - protein, 12 aa, artificial sequence
  Synthetic construct
HGHMVSTSQLSI

SEQ ID NO: 184 - protein, 7 aa, artificial sequence
  Synthetic construct
LSPSRMK

SEQ ID NO: 185 - protein, 7 aa, artificial sequence
  Synthetic construct
LPIPRMK

SEQ ID NO: 186 - protein, 7 aa, artificial sequence
  Synthetic construct
HQRPYLT

SEQ ID NO: 187 - protein, 7 aa, artificial sequence
  Synthetic construct
FPPLLRL

SEQ ID NO: 188 - protein, 7 aa, artificial sequence
  Synthetic construct
QATFMYN

SEQ ID NO: 189 - protein, 11 aa, artificial sequence
  Synthetic construct
VLTSQLPNHSM

SEQ ID NO: 190 - protein, 7 aa, artificial sequence
  Synthetic construct
HSTAYLT

SEQ ID NO: 191 - protein, 12 aa, artificial sequence
  Synthetic construct
APQQRPMKTFNT

SEQ ID NO: 192 - protein, 12 aa, artificial sequence
  Synthetic construct
APQQRPMKTVQY

SEQ ID NO: 193 - protein, 7 aa, artificial sequence
  Synthetic construct
PPWLDLL

SEQ ID NO: 194 - protein, 7 aa, artificial sequence
  Synthetic construct
PPWTFPL

SEQ ID NO: 195 - protein, 7 aa, artificial sequence
  Synthetic construct
SVTHLTS

SEQ ID NO: 196 - protein, 7 aa, artificial sequence
  Synthetic construct
VITRLTS

SEQ ID NO: 197 - protein, 12 aa, artificial sequence
  Synthetic construct
DLKPPLLALSKV

SEQ ID NO: 198 - protein, 12 aa, artificial sequence
  Synthetic construct
SHPSGALQEGTF

SEQ ID NO: 199 - protein, 12 aa, artificial sequence
  Synthetic construct
FPLTSKPSGACT

SEQ ID NO: 200 - protein, 12 aa, artificial sequence
  Synthetic construct
DLKPPLLALSKV

SEQ ID NO: 201 - protein, 7 aa, artificial sequence
  Synthetic construct
PLLALHS

SEQ ID NO: 202 - protein, 7 aa, artificial sequence
  Synthetic construct
VPISTQI

SEQ ID NO: 203 - protein, 12 aa, artificial sequence
  Synthetic construct
YAKQHYPISTFK

SEQ ID NO: 204 - protein, 7 aa, artificial sequence
  Synthetic construct
HSTAYLT

SEQ ID NO: 205 - protein, 12 aa, artificial sequence
  Synthetic construct
STAYLVAMSAAP

SEQ ID NO: 206 - protein, 12 aa, artificial sequence
  Synthetic construct
SVSVGMKPSPRP

SEQ ID NO: 207 - protein, 12 aa, artificial sequence
  Synthetic construct
TMGFTAPRFPHY

SEQ ID NO: 208 - protein, 12 aa, artificial sequence
  Synthetic construct
NLQHSVGTSPVW

SEQ ID NO: 209 - protein, 15 aa, artificial sequence
  Synthetic construct
QLSYHAYPQANHHAP

SEQ ID NO: 210 - protein, 12 aa, artificial sequence
  Synthetic construct
NQAASITKRVPY

SEQ ID NO: 211 - protein, 14 aa, artificial sequence
  Synthetic construct
SGCHLVYDNGFCDH

SEQ ID NO: 212 - protein, 14 aa, artificial sequence
  Synthetic construct
ASCPSASHADPCAH

SEQ ID NO: 213 - protein, 14 aa, artificial sequence
  Synthetic construct
NLCDSARDSPRCKV

SEQ ID NO: 214 - protein, 12 aa, artificial sequence
  Synthetic construct
NHSNWKTAADFL

SEQ ID NO: 215 - protein, 12 aa, artificial sequence
  Synthetic construct
GSSTVGRPLSYE

SEQ ID NO: 216 - protein, 12 aa, artificial sequence
  Synthetic construct
SDTISRLHVSMT

SEQ ID NO: 217 - protein, 12 aa, artificial sequence
  Synthetic construct
SPLTVPYERKLL

SEQ ID NO: 218 - protein, 12 aa, artificial sequence
  Synthetic construct
SPYPSWSTPAGR

SEQ ID NO: 219 - protein, 12 aa, artificial sequence
  Synthetic construct
VQPITNTRYEGG

SEQ ID NO: 220 - protein, 12 aa, artificial sequence
  Synthetic construct
WPMHPEKGSRWS

SEQ ID NO: 221 - protein, 14 aa, artificial sequence
  Synthetic construct
DACSGNGHPNNCDR

SEQ ID NO: 222 - protein, 14 aa, artificial sequence
  Synthetic construct
DHCLGRQLQPVCYP

SEQ ID NO: 223 - protein, 14 aa, artificial sequence
  Synthetic construct
DWCDTIIPGRTCHG

SEQ ID NO: 224 - protein, 12 aa, artificial sequence
  Synthetic construct
ALPRIANTWSPS

SEQ ID NO: 225 - protein, 12 aa, artificial sequence
  Synthetic construct
YPSFSPTYRPAF

SEQ ID NO: 226 - protein, 20 aa, artificial sequence
  Synthetic construct
AHPESLGIKYALDGNSDPHA

SEQ ID NO: 227 - protein, 20 aa, artificial sequence
  Synthetic construct
ASVSNYPPIHHLATSNTTVN

SEQ ID NO: 228 - protein, 14 aa, artificial sequence
  Synthetic construct
DECMEPLNAAHCWR

SEQ ID NO: 229 - protein, 14 aa, artificial sequence
  Synthetic construct
DECMHGSDVEFCTS

SEQ ID NO: 230 - protein, 14 aa, artificial sequence
  Synthetic construct
DLCSMQMMNTGCHY

SEQ ID NO: 231 - protein, 14 aa, artificial sequence
  Synthetic construct
DLCSSPSTWGSCIR

SEQ ID NO: 232 - protein, 20 aa, artificial sequence
  Synthetic construct
DPNESNYENATTVSQPTRHL

SEQ ID NO: 233 - protein, 20 aa, artificial sequence
  Synthetic construct
EPTHPTMRAQMHQSLRSSSP

SEQ ID NO: 234 - protein, 20 aa, artificial sequence
  Synthetic construct
GNTDTTPPNAVMEPTVQHKW

SEQ ID NO: 235 - protein, 15 aa, artificial sequence
  Synthetic construct
NGPDMVQSVGKHKNS

SEQ ID NO: 236 - protein, 15 aa, artificial sequence
  Synthetic construct
NGPEVRQIPANFEKL

SEQ ID NO: 237 - protein, 20 aa, artificial sequence
  Synthetic construct
NNTSADNPPETDSKHHLSMS

SEQ ID NO: 238 - protein, 20 aa, artificial sequence
  Synthetic construct
NNTWPEGAGHTMPSTNIRQA

SEQ ID NO: 239 - protein, 20 aa, artificial sequence
  Synthetic construct
NPTATPHMKDPMHSNAHSSA

SEQ ID NO: 240 - protein, 20 aa, artificial sequence
  Synthetic construct
NPTDHIPANSTNSRVSKGNT

SEQ ID NO: 241 - protein, 15 aa, artificial sequence
  Synthetic construct
NPTDSTHMMHARNHE

SEQ ID NO: 242 - protein, 14 aa, artificial sequence
  Synthetic construct
QHCITERLHPPCTK

SEQ ID NO: 243 - protein, 14 aa, artificial sequence
  Synthetic construct
TPCAPASFNPHCSR

SEQ ID NO: 244 - protein, 14 aa, artificial sequence
  Synthetic construct
TPCATYPHFSGCRA

SEQ ID NO: 245 - protein, 20 aa, artificial sequence
  Synthetic construct
WCTDFCTRSTPTSTSRSTTS

SEQ ID NO: 246 - protein, 20 aa, artificial sequence
  Synthetic construct
APPLKTYMQERELTMSQNKD

SEQ ID NO: 247 - protein, 20 aa, artificial sequence
  Synthetic construct
EPPTRTRVNNHTVTVQAQQH

SEQ ID NO: 248 - protein, 14 aa, artificial sequence
  Synthetic construct
GYCLRGDEPAVCSG

SEQ ID NO: 249 - protein, 20 aa, artificial sequence
  Synthetic construct
LSSKDFGVTNTDQRTYDYTT

SEQ ID NO: 250 - protein, 14 aa, artificial sequence
  Synthetic construct
NFCETQLDLSVCTV

SEQ ID NO: 251 - protein, 14 aa, artificial sequence
  Synthetic construct
NTCQPTKNATPCSA

SEQ ID NO: 252 - protein, 20 aa, artificial sequence
  Synthetic construct
PSEPERRDRNIAANAGRFNT

SEQ ID NO: 253 - protein, 18 aa, artificial sequence
  Synthetic construct
THNMSHFPPSGHPKRTAT

SEQ ID NO: 254 - protein, 14 aa, artificial sequence
  Synthetic construct
TTCPTMGTYHVCWL

SEQ ID NO: 255 - protein, 20 aa, artificial sequence
  Synthetic construct
YCADHTPDPANPNKICGYSH

SEQ ID NO: 256 - protein, 20 aa, artificial sequence
  Synthetic construct
AANPHTEWDRDAFQLAMPPK

SEQ ID NO: 257 - protein, 20 aa, artificial sequence
  Synthetic construct
DLHPMDPSNKRPDNPSDLHT

SEQ ID NO: 258 - protein, 14 aa, artificial sequence
  Synthetic construct
ESCVSNALMNQCIY

SEQ ID NO: 259 - protein, 20 aa, artificial sequence
  Synthetic construct
HNKADSWDPDLPPHAGMSLG

SEQ ID NO: 260 - protein, 20 aa, artificial sequence
  Synthetic construct
LNDQRKPGPPTMPTHSPAVG

SEQ ID NO: 261 - protein, 14 aa, artificial sequence
  Synthetic construct
NTCATSPNSYTCSN

SEQ ID NO: 262 - protein, 14 aa, artificial sequence
  Synthetic construct
SDCTAGLVPPLCAT

SEQ ID NO: 263 - protein, 20 aa, artificial sequence
  Synthetic construct
TIESSQHSRTHQQNYGSTKT

SEQ ID NO: 264 - protein, 20 aa, artificial sequence
  Synthetic construct
VGTMKQHPTTTQPPRVSATN

SEQ ID NO: 265 - protein, 20 aa, artificial sequence
  Synthetic construct
YSETPNDQKPNPHYKVSGTK

SEQ ID NO: 266 - protein, 8 aa, Artificial Sequence
  Caspase 3 cleavage site
LESGDEVD

SEQ ID NO: 267 - protein, 37 aa, Artificial Sequence
  Peptide Spacer
TSTSKASTTTTSSKTTTTSSKTTTTTSKTSTTSSSST

SEQ ID NO: 268 - protein, 22 aa, Artificial Sequence
  Peptide Spacer
GQGGYGGLGSQGAGRGGLGGQG

SEQ ID NO: 269 - protein, 10 aa, Artificial Sequence
  Peptide Spacer
GPGGYGPGQQ